(19) United States
(12) Patent Application Publication
(10) Pub. No.: US 2025/0353834 A1
(43) Pub. Date: Nov. 20, 2025

(54) PROCESS FOR THE SYNTHESIS OF SUBSTITUTED TETRAHYDROFURAN MODULATORS OF SODIUM CHANNELS

(71) Applicant: VERTEX PHARMACEUTICALS INCORPORATED, Boston, MA (US)

(72) Inventors: Cristian HARRISON, Beverly, MA (US); Christopher John DAVIS, Salisbury, Wiltshire (GB); Simon Robert Everitt, Beaconsfield, Bucks (GB); Enrico EMER, Oxford, Oxfordshire (GB); Michael Edward O'Donnell, Abingdon, Oxfordshire (GB); Stephen SHANAHAN, Wokingham, Berkshire (GB); Mireia Sidera PORTELA, Abingdon, Oxfordshire (GB); Bruno Artur SOUSA, Reading, Berkshire (GB); Shujauddin M. CHANGI, Needham, MA (US); Berenice L. LEWANDOWSKI, Braintree, MA (US); Maninder PANESAR, Didcot, Oxfordshire (GB); Andrew MCTIERNAN, Framingham, MA (US); Tharanga K. WIJETHUNGA, Quincy, MA (US)

(73) Assignee: VERTEX PHARMACEUTICALS INCORPORATED, Boston, MA (US)

(21) Appl. No.: 18/566,170

(22) PCT Filed: Jun. 3, 2022
(86) PCT No.: PCT/US2022/032167

§ 371 Date: Dec. 1, 2023

Related U.S. Application Data
(60) Provisional application No. 63/196,868, filed on Jun. 4, 2021.

(51) Int. Cl. C07D 405/12; C07D 307/20; C07D 307/24; C07D 307/58; C12P 17/04

(52) U.S. Cl. CPC C07D 405/12; C07D 307/20; C07D 307/24; C07D 307/58; C12P 17/04

(57) ABSTRACT

Provided in this application is a process for making Compound I (I) and pharmaceutically acceptable salts thereof, useful as inhibitors of sodium channels. Processes for making various intermediate products, and suitable salts thereof, are also provided.

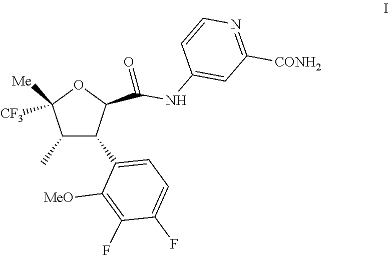 [SMILES: COc1c([C@H]2[C@H](C(=O)Nc3ccnc(C(N)=O)c3)O[C@@](C)(C(F)(F)F)[C@H]2C)ccc(F)c1F]

### CROSS REFERENCE TO RELATED APPLICATIONS

This application claim benefit of U.S. Provisional Patent Application No. 63/196,868, filed Jun. 4, 2021, which is incorporated by reference herein in its entirety.

### BACKGROUND

Pain is a protective mechanism that allows healthy animals to avoid tissue damage and to prevent further damage to injured tissue. Nonetheless there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137 (3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain.

Voltage-gated sodium channels (Naᵥs) are involved in pain signaling. Naᵥs are biological mediators of electrical signaling as they mediate the rapid upstroke of the action potential of many excitable cell types (e.g. neurons, skeletal myocytes, cardiac myocytes). The evidence for the role of these channels in normal physiology, the pathological states arising from mutations in sodium channel genes, preclinical work in animal models, and the clinical pharmacology of known sodium channel modulating agents all point to the central role of Naᵥs in pain sensation (Rush, A. M. and T. R. Cummins, Painful Research: Identification of a Small-Molecule Inhibitor that Selectively Targets Naᵥ1.8 Sodium Channels. Mol. Interv., 2007. 7 (4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Naᵥs mediate the rapid upstroke of the action potential of many excitable cell types (e.g. neurons, skeletal myocytes, cardiac myocytes), and thus are involved in the initiation of signaling in those cells (Hille, Bertil, Ion Channels of Excitable Membranes, Third ed. (Sinauer Associates, Inc., Sunderland, MA, 2001)). Because of the role Naᵥs play in the initiation and propagation of neuronal signals, antagonists that reduce Naᵥ currents can prevent or reduce neural signaling and Naᵥ channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Naᵥ channels. The local anesthetic drugs such as lidocaine block pain by inhibiting Naᵥ channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J. Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Naᵥs form a subfamily of the voltage-gated ion channel super-family and comprises 9 isoforms, designated Naᵥ1.1-Naᵥ1.9. The tissue localizations of the nine isoforms vary. Naᵥ1.4 is the primary sodium channel of skeletal muscle, and Naᵥ1.5 is primary sodium channel of cardiac myocytes. Naᵥs 1.7, 1.8 and 1.9 are primarily localized to the peripheral nervous system, while Naᵥs 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Naᵥ1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379 (6562): p. 257-62). Since then, Naᵥ1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22 (23): p. 10277-90). Naᵥ1.8 is involved in spontaneous firing in damaged neurons, like those that drive neuropathic pain (Roza, C., et al., The tetrodotoxin-resistant Na⁺ channel Naᵥ1.8 is essential for the expression of spontaneous activity in damaged sensory axons of mice. J. Physiol., 2003. 550 (Pt 3): p. 921-6; Jarvis, M. F., et al., A-803467, a potent and selective Naᵥ1.8 sodium channel blocker, attenuates neuropathic and inflammatory pain in the rat. Proc. Natl. Acad. Sci. USA, 2007. 104 (20): p. 8520-5; Joshi, S. K., et al., Involvement of the TTX-resistant sodium channel Naᵥ1.8 in inflammatory and neuropathic, but not post-operative, pain states. Pain, 2006. 123 (1-2): pp. 75-82; Lai, J., et al., Inhibition of neuropathic pain by decreased expression of the tetrodotoxin-resistant sodium channel, Naᵥ1.8. Pain, 2002. 95 (1-2): p. 143-52; Dong, X. W., et al., Small interfering RNA-mediated selective knockdown of Naᵥ1.8 tetrodotoxin-resistant sodium channel reverses mechanical allodynia in neuropathic rats. Neuroscience, 2007. 146 (2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64 (6): p. 644-53; Coward, K., et al., Immunolocalization of SNS/PN3 and NaN/SNS2 sodium channels in human pain states. Pain, 2000. 85 (1-2): p. 41-50; Yiangou, Y., et al., SNS/PN3 and SNS2/NaN sodium channel-like immunoreactivity in human adult and neonate injured sensory nerves. FEBS Lett., 2000. 467 (2-3): p. 249-52; Ruangsri, S., et al., Relationship of axonal voltage-gated sodium channel 1.8 (Naᵥ1.8) mRNA accumulation to sciatic nerve injury-induced painful neuropathy in rats. J. Biol. Chem. 286(46): p. 39836-47). The small DRG neurons where Naᵥ1.8 is expressed include the nociceptors involved in pain signaling. Naᵥ1.8 mediates large amplitude action potentials in small neurons of the dorsal root ganglia (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22 (23): p. 10277-90). Naᵥ1.8 is necessary for rapid repetitive action potentials in nociceptors, and for spontaneous activity of damaged neurons. (Choi, J. S. and S. G. Waxman, Physiological interactions between Naᵥ1.7 and Naᵥ1.8 sodium channels: a computer simulation study. J. Neurophysiol. 106(6): p. 3173-84; Renganathan, M., T. R. Cummins, and S. G. Waxman, Contribution of Na (v)1.8 sodium channels to action potential electrogenesis in DRG neurons. J. Neurophysiol., 2001. 86 (2): p. 629-40; Roza, C., et al., The tetrodotoxin-resistant Na⁺ channel Naᵥ1.8 is essential for the expression of spontaneous activity in damaged sensory axons of mice. J. Physiol., 2003. 550 (Pt 3): p. 921-6). In depolarized or damaged DRG neurons, Naᵥ1.8 appears to be a driver of hyper-excitablility (Rush, A. M., et al., A single sodium channel mutation produces hyper- or hypoexcitability in different types of neurons. Proc. Natd. Acad. Sci. USA, 2006. 103 (21): p. 8245-50). In some animal pain models, Naᵥ1.8 mRNA expression levels have been shown to increase in the DRG (Sun, W., et al., Reduced conduction failure of the main axon of polymodal nociceptive C-fibers contributes to painful diabetic neuropathy in rats. Brain, 135 (Pt 2): p. 359-75; Strickland, I. T., et al., Changes in the expression of Naᵥ1.7, Naᵥ1.8 and Naᵥ1.9 in a distinct population of dorsal root ganglia innervating the rat knee joint in a model of chronic inflammatory joint pain. Eur. J. Pain, 2008. 12 (5): p. 564-72; Qiu, F., et al., Increased expression of tetrodotoxin-resistant sodium channels Naᵥ1.8 and Naᵥ1.9 within dorsal root ganglia in a rat model of bone cancer pain. Neurosci. Lett., 512(2): p. 61-6).

The primary drawback to some known Naᵥ inhibitors is their poor therapeutic window, and this is likely a consequence of their lack of isoform selectivity. Since Naᵥ1.8 is primarily restricted to the neurons that sense pain, selective Naᵥ1.8 blockers are unlikely to induce the adverse events common to non-selective Naᵥ blockers. Accordingly, there remains a need to develop additional Naᵥ channel modulators, preferably those that are highly potent and selective for Naᵥ1.8.

### SUMMARY

In one aspect, the invention relates to a method of preparing a compound of formula I

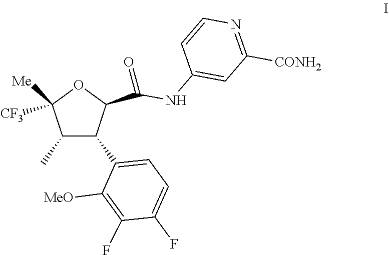 [SMILES: COc1c([C@H]2[C@H](C(=O)Nc3ccnc(C(N)=O)c3)O[C@@](C)(C(F)(F)F)[C@H]2C)ccc(F)c1F]

or a pharmaceutically acceptable salt thereof.

In a second embodiment, the method comprises converting any of compounds of formulae II-V and VII-XXI to the compound of formula I following the reaction steps described herein.

### BRIEF DESCRIPTION OF THE DRAWINGS

FIG. 1 shows the X-ray Diffraction Pattern of Form A of Compound III AMB salt.

FIG. 2 shows the X-ray Diffraction Pattern of Form A of Quinine salt of Formula IV.

### DETAILED DESCRIPTION

In one embodiment, the skilled artisan could start with any compounds of formulae II-V and VII-XXI to prepare the compound of formula I or any of the intermediate compounds of formulae IT-V and VII-XX by following the reactions illustrated in Schemes 1 and 2.

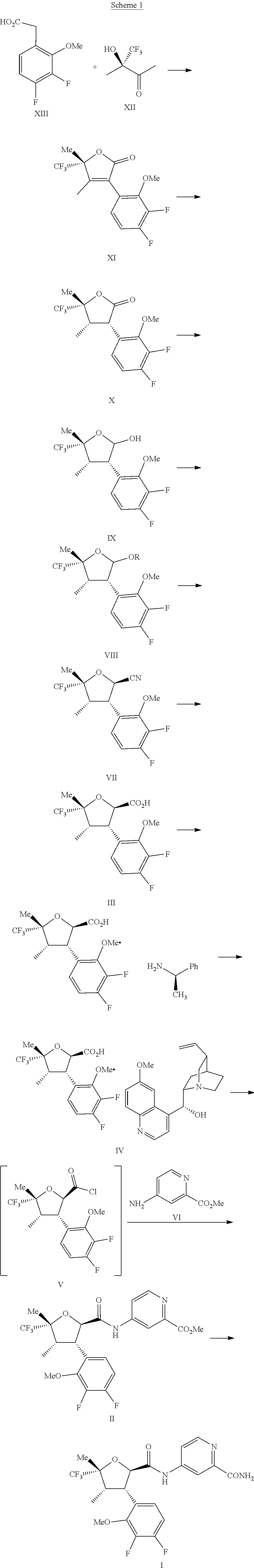

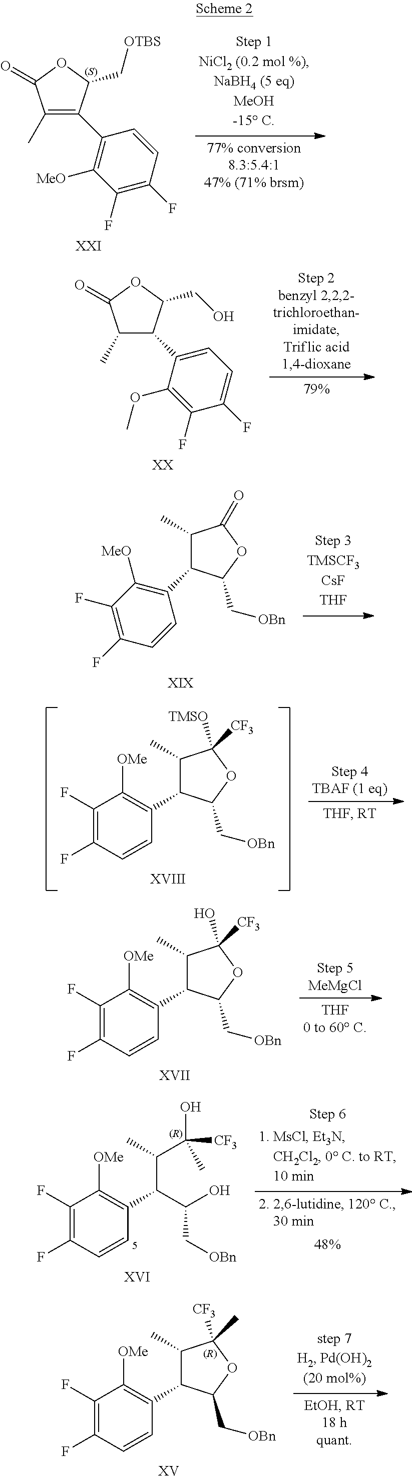

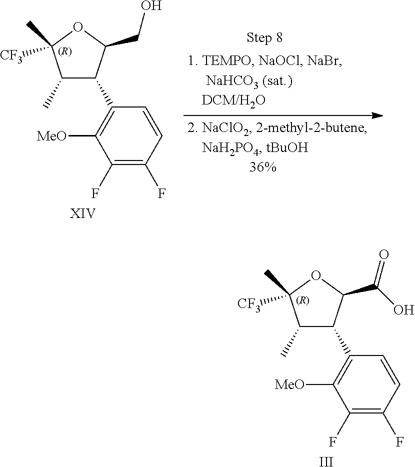

The method steps described herein may refer to conversion of a starting compound of formulae II-V and VII-XXI to the compound of formula I. The skilled artisan would understand that such methods can also be used to prepare any intermediate between any starting compound and the compound of formula I. For example, conversion of the compound of formula III to the compound of formula I goes through intermediate compounds II, IV, and V. As such, the skilled artisan would understand that the methods described for converting the compound of formula III to the compound of formula I can be used to prepare any of intermediate compounds II, IV, and V from the compound of formula III. Similarly, conversion of the compound of formula IX to the compound of formula I goes through preparation of intermediate compounds II-V, VII, and VIII. As such, the skilled artisan would understand that the methods described for converting the compound of formula IX to the compound of formula I can be used to prepare any of intermediate compounds II-V, VII, and VIII starting with the compound of formula IX or any intermediate compound can be converted to the desired intermediate compound using the methods described herein. Thus, the present application contemplates preparing intermediate compounds II-V and VII-XXI starting with any intermediate or starting material that precedes the intermediate that is being prepared. For example, intermediate compound II may be prepared starting with any of compounds III-V and VII-XXI. Similarly, compound VII may be prepared starting with any of compounds VIII-XXI.

In one embodiment, the present application provides a method for converting a compound of formula III,

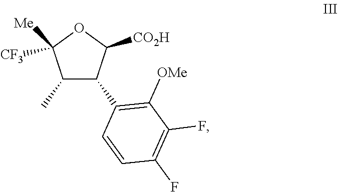 [SMILES: COc1c([C@H]2[C@H](C(=O)O)O[C@@](C)(C(F)(F)F)[C@H]2C)ccc(F)c1F]

or a salt thereof, to the compound of formula I.

In some embodiments, the method of converting the compound of formula III to the compound of formula I comprises preparing the compound of formula IV:

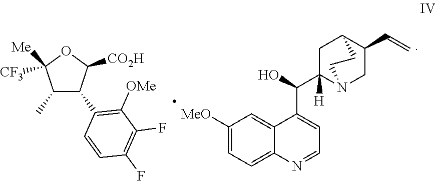 [SMILES: COc1c([C@H]2[C@H](C(=O)O)O[C@@](C)(C(F)(F)F)[C@H]2C)ccc(F)c1F ; C=C[C@H]1C[N@]2CC[C@H]1C[C@H]2[C@H](O)c1ccnc2ccc(OC)cc12]

The compound of formula IV may be prepared directly from the compound of formula III by reacting the compound of formula III with quinine in a solvent comprising a polar solvent. In some embodiments, the compound of formula IV may be prepared by dissolving or suspending the compound of formula III and quinine in a solvent comprising a polar solvent. In some emboiments, the solvent comprises DCM and heptane; toluene, EtOAc and Heptane; MTBE; acetonitrile and heptane; 2-MeTHF and heptane, or MEK and heptane. In other embodiments, the solvent comprises DCM, heptane, toluene, EtOAc, MTBE, acetonitrile, 2-MeTHF, or MEK.

In some embodiments, the compound of formula IV is prepared by first converting the compound of formula III to a salt (for example, a salt of the compound of formula III with 1-phenylethylamine) followed by conversion of such salt to the quinine salt using any method known to those skilled in the art. Further, the salt of compound III (e.g., 1-phenylethylamine salt of the compound of formula III) may be converted first to the free base before converting the latter to the quinine salt of the compound of formula III (i.e., the compound of formula IV):

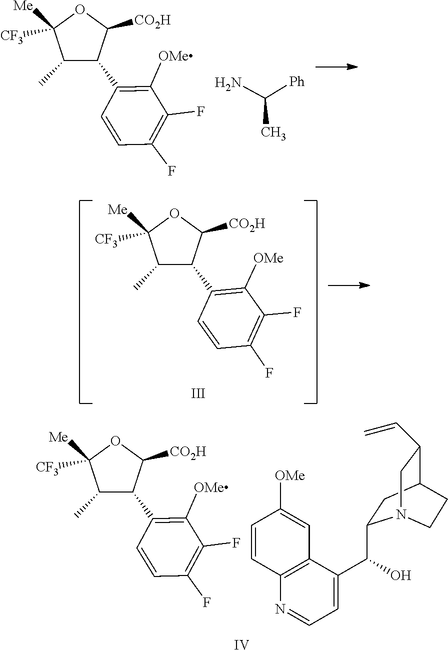

Compound III may be converted to compound I via an esterification reaction between compounds III and VI. The esterification reaction may be conducted via an intermediate compound of formula V. Alternatively, the esterification of compound III with compound VI to afford compound II may be conducted via a coupling agent and without the use of a chlorinating agent.

In some embodiments, the method of converting the compound of formula III to the compound of formula I comprises reacting the compound of formula III or a salt thereof (such as the compound of formula IV or (R)-1-phenylethylamine salt of the compound of formula III) with a chlorinating agent to afford a compound of formula V:

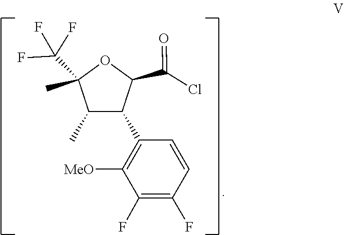

In the compound of formula V, the parentheticals around the compound indicate that the compound of formula V may not be isolated.

A mixture of the compounds of formulae III and IV may also be converted to the compound of formula II via a coupling reaction that may or may not go through a compound of formula V. In some embodiments, the mixture is first converted to the compound of formula V followed by a reaction between compounds of formulae V and VI as described elsewhere in this application. In other embodiments, the mixture of the compounds of formulae III and IV may be converted to the compound of formula II via a coupling reaction that includes a step in which the compound of formula IV in the mixture is first converted to a free acid of formula III before coupling the acid with the compound of formula VI.

Any chlorinating agent suitable for chlorinating compound III, or a salt thereof, may be used. In some embodiments, the chlorinating agent is thionyl chloride, methanesulfonyl chloride, phosphorus oxychloride, phosphorus pentachloride, phosgene, oxalyl chloride, isobutyl chloroformate (IBCF), pivaloyl chloride (PivCl), or diphenylphosphinic chloride (DPPCl). In some embodiments, the chlorinating agent is phosgene.

The reaction between compound III and the chlorinating agent may be conducted in the presence of a non-nucleophilic base. Any suitable non-nucleophilic base may be used to scavenge the HCl generated by the chlorinating reaction.

Suitable non-nucleophilic bases are typically tertiary or aromatic amines where the nitrogen of the amine base does not carry an H atom. The non-nucleophilic base may be bulky bases that are non-nucleophilic because of steric hindrance. Examples of suitable bases include Hunig's base, triethylamine, diisopropyl ethylamine, N-methylmorpholine, 1,8-diazabicyclo[5.4.0]undec-7-ene, pyridine, butylamine, or 1,5-diazabicyclo(4.3.0)non-5-ene, or a mixture thereof. In some embodiments, the reaction between compound III and the chlorinating agent is conducted at a temperature of no more than about 90° C. In some embodiments, the esterification reaction between compound III, or a salt thereof, and compound of formula VI may be conducted at a temperature of no more than about 60° C., about 70° C., or about 80° C. In other embodiments, the esterification reaction between compound III, or a salt thereof, and compound of formula VI may be conducted at a temperature of no more than about 70° C.

In a further embodiment, the method of converting the compound of formula III to the compound of formula I comprises halogenating the compound of formula III or IV to afford the compound of formula V followed by esterification of the compound of formula V with a compound of formula VI:

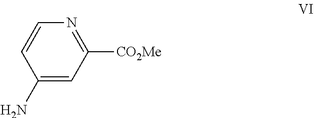 [SMILES: COC(=O)c1cc(N)ccn1]

to afford a compound of formula II:

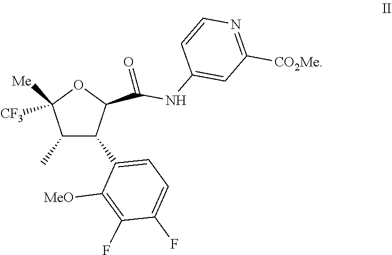 [SMILES: COC(=O)c1cc(NC(=O)[C@@H]2O[C@@](C)(C(F)(F)F)[C@@H](C)[C@H]2c2ccc(F)c(F)c2OC)ccn1]

The esterification reaction may be conducted in a solvent comprising DCM, toluene, MeCN, EtOAc, 2-methyl THF, CH₂Cl₂, IPAc, or a mixture thereof. The esterification reaction may be conducted in the presence of 1,1′-carbonyldiimidazole (CDI), 1-ethyl-3-(3-dimethylaminopropyl)carbodiimide (EDCl), or propylphosphonic anhydride (T3P). In some embodiments, the esterification reaction may be conducted in the presence of a base selected from the group consisting of trimethylamine, N-methylimidazole, pyridine, 4-methylmorpholine, Hunig's base, DABCO, and NaOH, and the like. In further embodiments, the base may be any C1-C4 alkyl tertiary amine, such as triethyl amine, ethyldimethyl amine, ethyldipropyl amine and various alkyl combinations thereof.

After completion of the esterification reaction (via an acid chloride of formula V or directly between compounds III and VI using a coupling agent), the compound of formula II may be purified, for example, by recrystallizing it from a solvent comprising methanol or water or a mixture thereof. Other suitable combination of two solvents include ethanol/water, toluene/heptane, IPA/water, etc. In any of these combinations, the compound of formula II is dissolved in one solvent at boiling or near boiling temperature followed by addition of the second solvent until the solution becomes turbid. The turbid suspension is allowed to cool down to room temperature (or cooled with an ice bath) followed by filtration of the solid.

In some embodiments, the method of converting the compound of formula III to the compound of formula I further comprises an amidation reaction comprising reacting the compound of formula II with ammonia to afford the compound of formula I. In some embodiments, the amidation reaction may be conducted in a solvent. In some embodiments, the solvent is methanol, ethanol, IPA, MeCN, THF, 2-MeTHF, water, or a mixture thereof. Amidation of the compound of formula II to afford a compound of formula I may be conducted in the presence of a weak, non-nucleophilic base. Examples of bases suitable as additives to the amidation reaction include Mg(OMe)₂, CaCl₂), DIPEA, and K₂CO₃.

The amidation reaction may be conducted using a solution of ammonia in the reaction solvent, ammonia in gas form (i.e., by bubbling ammonia gas into the reaction solution), or in the form of ammonium hydroxide or ammonium salt (such as chloride) where ammonia is generated in situ (e.g., by neutralizing ammonium hydroxide with an acid).

The compound of formula I may be recrystallized from a solvent system comprising acetone to afford the compound of formula I as a solid. In some embodiments, the recrystallization solvent system comprises acetone and water. In other embodiments, the recrystallization solvent may comprise IPA or the following pairs of solvents: ethyl acetate/heptane, IPA/water, ethanol/water, isopropyl acetate/heptane.

Although the skilled artisan may devise a method of making the compound of formula III used to prepare the compound of formula I, the inventors of the present application contemplate preparing the compound of formula III using the following process.

In one embodiment, the compound of formula III may be obtained by hydrolyzing a cyano-compound of formula VII:

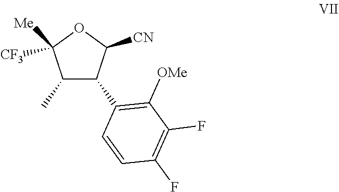 [SMILES: COc1c([C@H]2[C@H](C#N)O[C@@](C)(C(F)(F)F)[C@H]2C)ccc(F)c1F]

to afford the compound of formula III. Any base or acid suitable for hydrolyzing the CN group without affecting other functional groups in the compound of formula VII may be used. In one embodiment, a strong base (such as NaOH, KOH, and the like) or strong acid (HCl, sulfuric acid, or the like) may be used. In one embodiment, the CN group in the compound of formula VII is enzymatically hydrolyzed using a nitrilase. The CN hydrolysis of the compound of formula VII may be conducted in a solvent or solvent mixture. For example, ethanol, methanol, 1-propanol, 2-propanol, dioxane, water, THF, or a mixture thereof may be used. The hydrolysis reaction may be conducted at about 25-75° C., about 30-70° C., about 35-65° C., about 40-60° C., about 45-60° C., about 50-60° C., or about 55° C. As used in this paragraph, the term “about” in front of a temperature range applies to both ends of the range. It also means ±2.5° C.

The compound of formula VII may be obtained by reacting a compound of formula VIII,

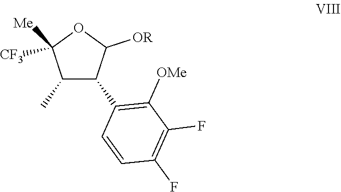

wherein OR is a leaving group;
with a cyanating agent (such as trimethylsilyl cyanide, diethylaluminum cyanide, KCN, NaCN, TBACN, HCN and the like) to afford the compound of formula VII. In one embodiment, the reaction between the cyanating agent (e.g., trimethylsilyl cyanide) and the compound of formula VIII may be conducted in the presence of a Lewis acid. In some embodiments, the Lewis acid is boron trifluoride ethyl etherate (BF₃OEt₂), TiCl₄, InCl₃, AgSbF₆, iodine, ZnBr₂, Al(OiPr)₃, MgCl₂, Mn(acac)₂, MnCl₂, TMSOTf, SnCl₄, ZnBr₂, Al(OiPr)₃, ZnCl₂, FeCl₂, Cu(NO₃)₂₆H₂O, Fe(OAc)₂, ScCl₃, and the like. In further embodiments, the Lewis acid is BF₃OEt₂. The cyanation reaction may be conducted in an organic solvent, for example toluene, dichloromethane, 2-methyl THF, acetonitrile, methanol, 1,2-dichloroethane, nitromethane, CPME, MTBE, DMAc, t-BuOAc, and the like.

In the compound of formula VIII, OR is a leaving group. In some embodiments, the leaving group OR on compound VIII is a group of formula OC(═O)—Z, OC(═O)OZ, OC(═O)CH═CH—Z, or OP(═O)Z₂ wherein Z may be an unsubstituted aryl or an aryl substituted by CN, halo, NO₂, or a short chain alkyl, alkoxy, haloalkyl, or haloalkoxy group wherein the short chain comprises 1, 2, 3, or 4 carbon atoms. Alternatively, Z is a short chain (i.e., with 1-4 carbon atoms) alkyl or haloalkyl group. Examples of aryl groups include phenyl and naphthyl.

The compound of formula IX may be converted to the compound of formula VII by introducing an R group to the compound of formula IX,

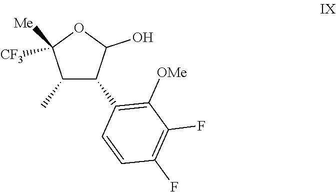 [SMILES: COc1c([C@H]2C(O)O[C@@](C)(C(F)(F)F)[C@H]2C)ccc(F)c1F]

in which the resulting compound (compound VIII) contains a leaving group OR. The skilled artisan would understand that the hydroxyl group of the compound of formula IX may be converted to any OR leaving group before replacing the OR group with CN.

In some embodiments, the compound of formula VIII may be obtained by reacting an alcohol of formula IX with an acid anhydride or an acid chloride to afford the compound of formula VIII. The compound of formula IX may be converted to a compound VIII by reacting a suitable acid chloride in the presence of a non-nucleophilic base (such as TEA, pyridine, Hunig's base, K₂CO₃, Na₂CO₃, NaHCO₃, 2,6-Lutidine, NMM, DABCO) in a polar solvent (such as toluene, cyclopentyl methyl ether (CPME), dichloromethane, dichloroethane, pyridine, chloroform, acetonitrile, THF, 2-MeTHF, EtOAc, IPAC or combinations thereof). Examples of esters (including carbonates) of the compound of formula VIII include:

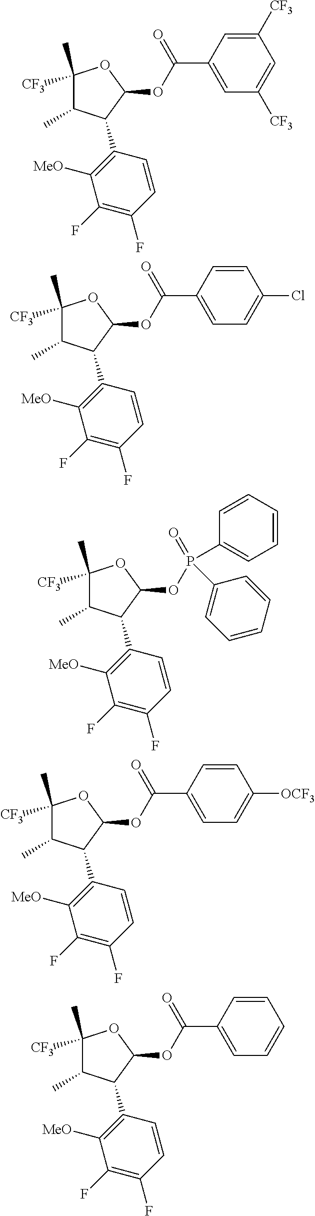

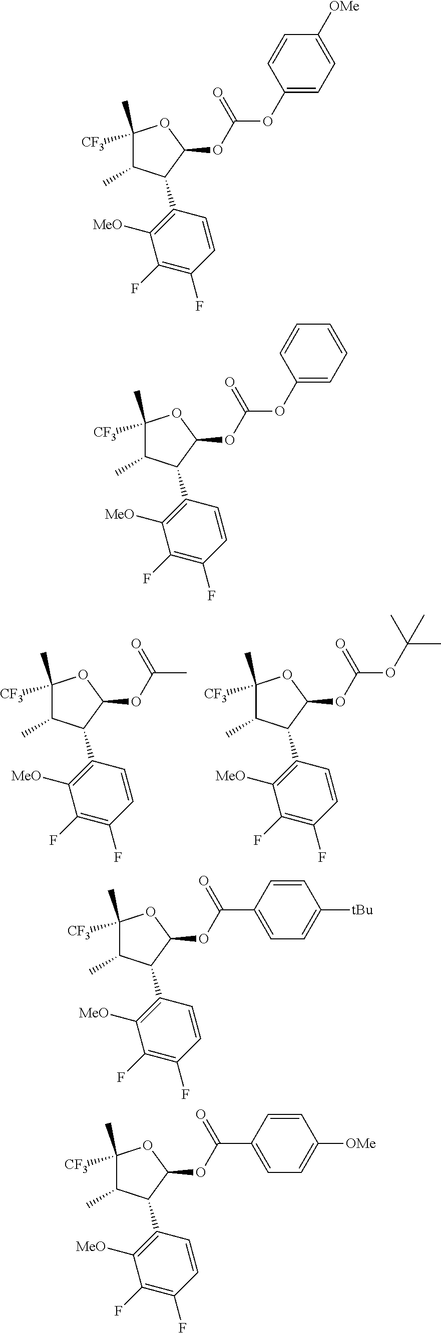

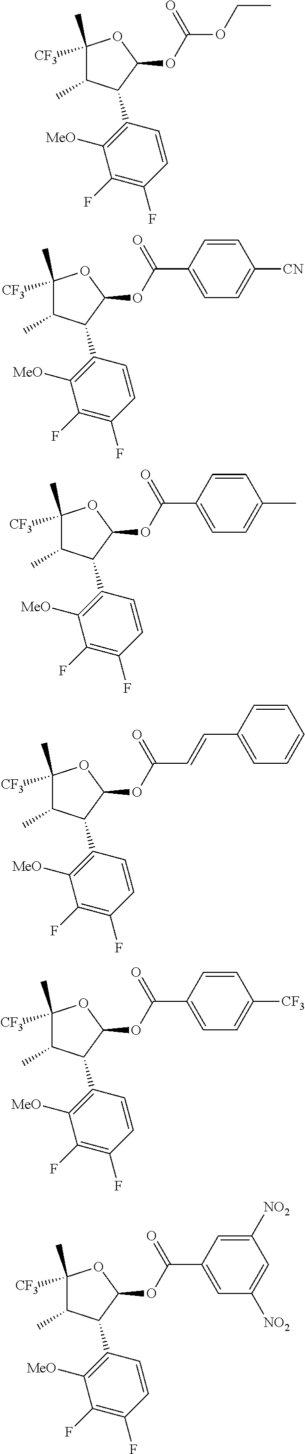

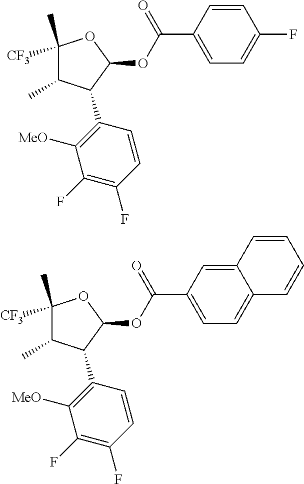 [SMILES: COc1c([C@H]2[C@H](OC(=O)c3ccc(F)cc3)O[C@@](C)(C(F)(F)F)[C@H]2C)ccc(F)c1F ; COc1c([C@H]2[C@H](OC(=O)c3ccc4ccccc4c3)O[C@@](C)(C(F)(F)F)[C@H]2C)ccc(F)c1F]

The compound of formula IX may be obtained by reducing a compound of formula X:

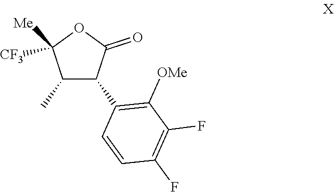 [SMILES: COc1c([C@H]2C(=O)O[C@@](C)(C(F)(F)F)[C@H]2C)ccc(F)c1F]

with a suitable reducing agent (e.g., diisobutylaluminum hydride, Red-A1, NaBH₄/BF₃, titanocene with polymethylhydrosiloxane or phenylsilane, super-hydride, L-selectride, Li(tBuO)₃AlH, and the like) to afford the compound of formula IX. The reduction reaction may be conducted in an organic solvent or solvent mixture. Suitable solvents include toluene, dichloromethane, 2-methyl THF, THF, TFT, MTBE, CPME, heptane, or a mixture thereof. The reaction may be conducted at below room temperature, for example, about −78° C. to 0° C., about −60° C. to 0° C., about −50° C. to −10° C., about −40° C. to −10° C., about −30° C. to −10° C., about −30° C. to −15° C., about −25° C. to −15° C., or about −20° C. The reduction reaction may be conducted in the presence of CuCl, CuI, CuTol, CuBr, CuF, Cu(II)Cl₂, DMAP, 2,6-lutidine, LiI, or pyridine.

The compound of formula X may be obtained via an asymmetric hydrogenation of a compound of formula XI,

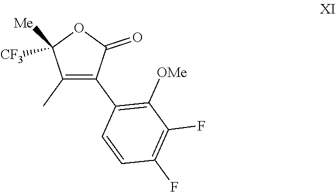 [SMILES: COc1c(C2=C(C)[C@](C)(C(F)(F)F)OC2=O)ccc(F)c1F]

to afford the compound of formula X. The asymmetric hydrogenation reaction may be catalyzed by any hydrogenation catalyst. Examples of hydrogenation catalyst include Pd/C, Pd/Al₂O₃, Pt/C, Pt/Si, Ni (Raney), Co (Raney), Rh/C, Ir/C, Ru/C, Pd(OH)₂, homogeneous chiral Ru and Rh using any suitable hydrogen source. Examples of suitable hydrogen sources include H₂ gas, NiCl₂/NaBH₄ in methanol, Et₃SiH, and the like. In some embodiments, hydrogen gas and Pd/C (catalyst) are used. The asymmetric hydrogenation reaction may be conducted in an organic solvent at between about 20 to 40 bar. A lower pressure may be use with high temperature and vice versa. For instance, about 5 bar may be suitable at about 40° C. Conversely, about 15-20 bar may be suitable at about 30° C. The skilled artisan can match pressure, temperature and reaction time to obtain desirable results. The asymmetric hydrogenation reaction may be conducted in an organic solvent or a solvent mixture. In one embodiment, the organic solvent is IPA, EtOAc, MeOH, nBuOH, THF, MTBE, CPME, IPAc, nBuAc, Toluene, Ethanol or a mixture thereof. The asymmetric hydrogenation reaction may be conducted in the presence of citric acid, benzoic acid, TFA, AcOH, H₂SO₄, H₃PO₄, MSA, Cs₂CO₃, CuCl, MgF₂, LiBr, CsF, ZnI, LiOTf, imidazole, KF, Bu₄NOAc, or NH₄BF₄.

In another embodiment, the compound of formula X may be obtained via hydrogenation of a compound of formula XI,

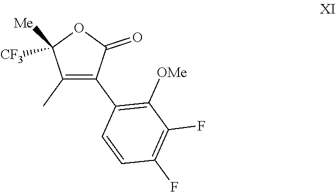 [SMILES: COc1c(C2=C(C)[C@](C)(C(F)(F)F)OC2=O)ccc(F)c1F]

to afford the compound of formula X. The hydrogenation reaction may be catalyzed by any hydrogenation catalyst. Examples of hydrogenation catalyst include Pd/C, Pd/Al₂O₃, Pt/C, Pt/Si, Ni (Raney), Co (Raney), Rh/C, Ir/C, Ru/C, Pd(OH)₂, using any suitable hydrogen source. Examples of suitable hydrogen sources include H₂ gas, NiCl₂/NaBH₄ in methanol, Et₃SiH, and the like. In some embodiments, hydrogen gas and Pd/C (catalyst) are used. The hydrogenation reaction may be conducted in an organic solvent at between about 20 to 40 bar. A lower pressure may be use with high temperature and vice versa. For instance, about 5 bar may be suitable at about 40° C. Conversely, about 15-20 bar may be suitable at about 30° C. The skilled artisan can match pressure, temperature and reaction time to obtain desirable results. The hydrogenation reaction may be conducted in an organic solvent or a solvent mixture. In one embodiment, the organic solvent is IPA, EtOAc, MeOH, nBuOH, THF, MTBE, CPME, IPAc, nBuAc, Toluene, Ethanol or a mixture thereof. The hydrogenation reaction may be conducted in the presence of citric acid, benzoic acid, TFA, AcOH, H₂SO₄, H₃PO₄, MSA, Cs₂CO₃, CuCl, MgF₂, LiBr, CsF, ZnI, LiOTf, imidazole, KF, Bu₄NOAc, or NH₄BF₄.

Alternatively, the compound of formula X may be prepared by coupling the compound of formula XXIV with 1-bromo-3,4-difluoro-2-methoxybenzene in the presence of a strong non-nucleophilic base (such as LiHMDS) and a Pd catalyst (such as bis(dibenzylideneacetone)palladium (0) in the presence of QPhos to afford a compound of formula XXIII wherein R1 is ═OMe, and R2 and R3 are F. See Scheme 3. Isomerization of the compound of formula XXIII affords the compound of formula III.

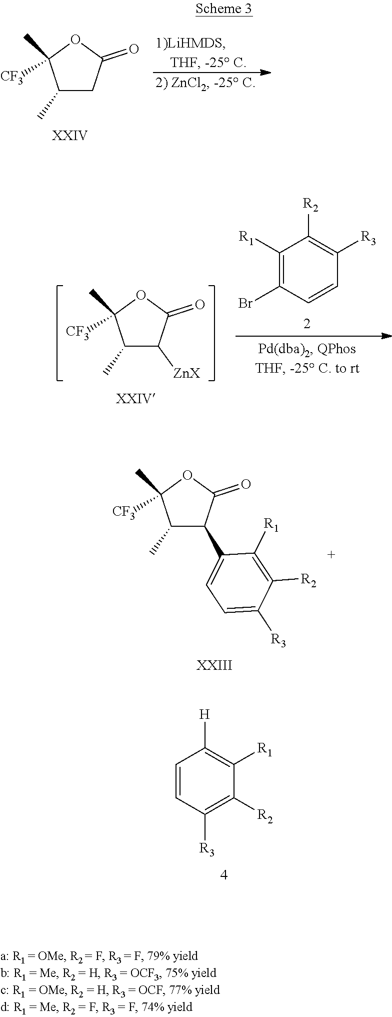

The compound of formula XI may be obtained by coupling a compound of formula XIII,

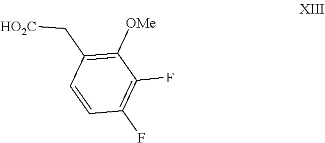 [SMILES: COc1c(CC(=O)O)ccc(F)c1F]

with a compound of formula XII,

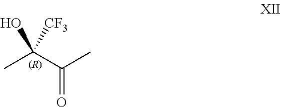

to afford the compound of formula XI. The coupling reaction between compounds of formulae XII and XIII is conducted in the presence of a coupling agent or a chlorinating agent. Examples of coupling agents suitable for the reactions between compounds of formulae XII and XIII include CDI, T3P, and the like. The coupling reaction between compounds of formulae XII and XIII may be conducted in the presence of a mild or a non-nucleophilic base. Examples of mild or non-nucleophilic bases suitable for the coupling reaction between compounds of formulae XII and XIII include imidazole, DIPEA, TEA, NMM, TBD, Na₂CO₃, K₃PO₄, DBU, DABCO, and MTBD. In some embodiments, the mild or non-nucleophilic base is imidazole, DIPEA, TEA, NMM, or TBD. The coupling reaction between compounds of formulae XII and XIII may be conducted in a polar aprotic solvent. Examples of polar aprotic solvents that may be suitable for the claims of the present application include solvents comprising MTBE, toluene, EtOAc, MeCN, THF, DMC, MeOAc, NMP, DMF, DMSO, THF, 2-MeTHF, and combinations thereof. The coupling reaction between compounds of formulae XII and XIII may be conducted at between about 20° C. and about 60° C., between about 25° C. and about 55° C., between about 30° C. and about 50° C., between about 30° C. and about 45° C., between about 30° C. and about 40° C., or about 35° C. As used in this paragraph, the term “about” means ±2.5° C.

When a chlorinating agent is used, the acid chloride of the compound of formula XIII is first prepared followed by the reaction of the acid chloride with the compound of formula XII. In this regard, the acid chloride of the compound of formula XIII need not be isolated before coupling it with the compound of formula XII. Examples of chlorinating agents suitable in coupling the compound of formula XII with the compound of formula XIII include oxalyl chloride, thionyl chloride, phosgene, and the like.

Alternatively, the compound of formula III may be prepared by oxidizing a compound of formula XIV,

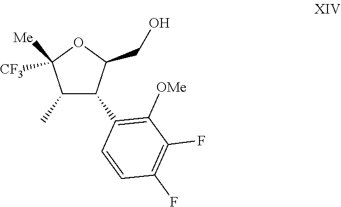 [SMILES: COc1c([C@H]2[C@H](CO)O[C@@](C)(C(F)(F)F)[C@H]2C)ccc(F)c1F]

to afford the compound of formula III.

The compound of formula XIV may be obtained by ring closure of a compound of formula XVI:

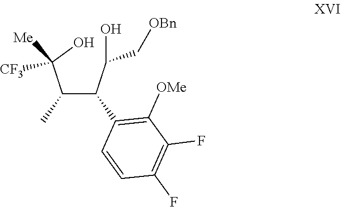 [SMILES: COc1c([C@@H]([C@H](O)COCc2ccccc2)[C@H](C)[C@@](C)(O)C(F)(F)F)ccc(F)c1F]

to afford the compound of formula XV;

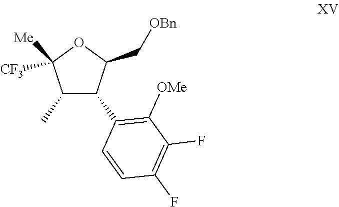 [SMILES: COc1c([C@H]2[C@H](COCc3ccccc3)O[C@@](C)(C(F)(F)F)[C@H]2C)ccc(F)c1F]

followed by deprotection of the compound of formula XV to afford the compound of formula XIV. In one embodiment, the ring closure reaction comprises reacting compound XVI with methanesulfonyl chloride or a similar chlorinating agent in the presence of a non-nucleophilic base. In one embodiment, the non-nucleophilic base is a tertiary amine. The ring closure reaction may be conducted at between about −5° C. and about 5° C. In some embodiments, the reaction is conducted between about −5° C. and about 5° C. Deprotection of compound XV may comprise reacting the compound of formula XV with H₂ in the presence of a hydrogenation catalyst (e.g., Pd/C catalyst or a similar catalyst) to afford the compound of formula XIV.

In one embodiment, the present application is directed to a method for preparing a compound of formula I, or a salt thereof, comprising converting a compound of formula IX using the methods described herein for converting the compound of formula IX to the compound of formula I.

Another embodiment of the present application is directed to a method for preparing a compound of formula I, or a salt thereof, comprising converting a compound of formula X using the methods described herein for converting the compound of formula X to the compound of formula I. Another embodiment of the present application is directed to a method for preparing a compound of formula I, or a salt thereof, comprising converting a compound of formula X using the methods described herein for converting the compound of formula X to the compound of formula I.

Another embodiment of the present application is directed to a method for preparing a compound of formula I, or a salt thereof, comprising converting a compound of formula XXI using the methods described herein for converting the compound of formula XXI to the compound of formula I.

Another embodiment of the present application is directed to a method for preparing a compound of formula I, or a salt thereof, comprising converting a compound of formula XX using the methods described herein for converting the compound of formula XX to the compound of formula I.

Another embodiment of the present application is directed to a method for preparing a compound of formula I, or a salt thereof, comprising converting a compound of formula XIX using the methods described herein for converting the compound of formula XIX to the compound of formula I.

Another embodiment of the present application is directed to a method for preparing a compound of formula I, or a salt thereof, comprising converting a compound of formula XVIII using the methods described herein for converting the compound of formula XVIII to the compound of formula I.

Another embodiment of the present application is directed to a method for preparing a compound of formula I, or a salt thereof, comprising converting a compound of formula XVII using the methods described herein for converting the compound of formula XVII to the compound of formula I.

Another embodiment of the present application is directed to a method for preparing a compound of formula I, or a salt thereof, comprising converting a compound of formula XVI using the methods described herein for converting the compound of formula XVI to the compound of formula I.

Another embodiment of the present application is directed to a method for preparing a compound of formula I, or a salt thereof, comprising converting a compound of formula XV using the methods described herein for converting the compound of formula XV to the compound of formula I.

Another embodiment of the present application is directed to a method for preparing a compound of formula I, or a salt thereof, comprising converting a compound of formula XIV using the methods described herein for converting the compound of formula XIV to the compound of formula I.

Another embodiment of the present application is directed to a method for preparing a compound of formula I, or a salt thereof, comprising converting a compound of formula XIII using the methods described herein for converting the compound of formula XIII to the compound of formula I.

Another embodiment of the present application is directed to a method for preparing a compound of formula I, or a salt thereof, comprising converting a compound of formula XII using the methods described herein for converting the compound of formula XII to the compound of formula I.

Another embodiment of the present application is directed to a method for preparing a compound of formula I, or a salt thereof, comprising converting a compound of formula XI using the methods described herein for converting the compound of formula XI to the compound of formula I.

Another embodiment of the present application is directed to a method for preparing a compound of formula I, or a salt thereof, comprising converting a compound of formula X using the methods described herein for converting the compound of formula X to the compound of formula I.

Another embodiment of the present application is directed to a method for preparing a compound of formula I, or a salt thereof, comprising converting a compound of formula IX using the methods described herein for converting the compound of formula IX to the compound of formula I.

Another embodiment of the present application is directed to a method for preparing a compound of formula I, or a salt thereof, comprising converting a compound of formula VIII using the methods described herein for converting the compound of formula VIII to the compound of formula I.

Another embodiment of the present application is directed to a method for preparing a compound of formula I, or a salt thereof, comprising converting a compound of formula VII using the methods described herein for converting the compound of formula VII to the compound of formula I.

Another embodiment of the present application is directed to a method for preparing a compound of formula I, or a salt thereof, comprising converting a compound of formula V using the methods described herein for converting the compound of formula V to the compound of formula I.

Another embodiment of the present application is directed to a method for preparing a compound of formula I, or a salt thereof, comprising converting a compound of formula IV using the methods described herein for converting the compound of formula IV to the compound of formula I.

Another embodiment of the present application is directed to a method for preparing a compound of formula I, or a salt thereof, comprising converting a compound of formula III using the methods described herein for converting the compound of formula III to the compound of formula I.

Another embodiment of the present application is directed to a method for preparing a compound of formula I, or a salt thereof, comprising converting a compound of formula II using the methods described herein for converting the compound of formula II to the compound of formula I.

In another embodiment, the present application is directed to a method for preparing an intermediate compound of formulae II-V and VII-XXI using the methods described herein.

In one embodiment, the present application is directed to a method for preparing a compound of formula II, or a salt thereof, comprising converting any of the compounds of formulae III-V and VII-XXI to the compound of formula II using the methods described herein.

In another embodiment, the present application is directed to a method for preparing a compound of formula III, or a salt thereof, comprising converting any of the compounds of formulae IV-V and VII-XXI to the compound of formula III using the methods described herein.

In another embodiment, the present application is directed to a method for preparing a compound of formula IV, or a salt thereof, comprising converting any of the compounds of formulae V and VII-XXI to the compound of formula IV using the methods described herein.

In another embodiment, the present application is directed to a method for preparing a compound of formula V, or a salt thereof, comprising converting any of the compounds of formulae VII-XXI to the compound of formula V using the methods described herein.

In another embodiment, the present application is directed to a method for preparing a compound of formula VII, or a salt thereof, comprising converting any of the compounds of formulae VIII-XXI to the compound of formula VII using the methods described herein.

In another embodiment, the present application is directed to a method for preparing a compound of formula VIII, or a salt thereof, comprising converting any of the compounds of formulae IX-XXI to the compound of formula VIII using the methods described herein.

In another embodiment, the present application is directed to a method for preparing a compound of formula IX, or a salt thereof, comprising converting any of the compounds of formulae X-XXI to the compound of formula IX using the methods described herein.

In another embodiment, the present application is directed to a method for preparing a compound of formula X, or a salt thereof, comprising converting any of the compounds of formulae XI-XXI to the compound of formula X using the methods described herein.

In another embodiment, the present application is directed to a method for preparing a compound of formula XI, or a salt thereof, comprising converting any of the compound of formula XII-XXI to the compound of formula XI using the methods described herein.

In another embodiment, the present application is directed to a method for preparing a compound of formula XII, or a salt thereof, comprising converting any of the compounds of formulae XIII-XXI to the compound of formula XII using the methods described herein.

In another embodiment, the present application is directed to a method for preparing a compound of formula XIII, or a salt thereof, comprising converting any of the compounds of formulae XIV-XXI to the compound of formula XIII using the methods described herein.

In another embodiment, the present application is directed to a method for preparing a compound of formula XIV, or a salt thereof, comprising converting any of the compounds of formulae XV-XXI to the compound of formula XIV using the methods described herein.

In another embodiment, the present application is directed to a method for preparing a compound of formula XV, or a salt thereof, comprising converting any of the compounds of formulae XVI-XXI to the compound of formula XV using the methods described herein.

In another embodiment, the present application is directed to a method for preparing a compound of formula XVI, or a salt thereof, comprising converting any of the compounds of formulae XVII-XXI to the compound of formula XVI using the methods described herein.

In another embodiment, the present application is directed to a method for preparing a compound of formula XVII, or a salt thereof, comprising converting any of the compounds of formulae XVIII-XXI to the compound of formula XVII using the methods described herein.

In another embodiment, the present application is directed to a method for preparing a compound of formula XVIII, or a salt thereof, comprising converting any of the compounds of formulae XIX-XXI to the compound of formula XVIII using the methods described herein.

In another embodiment, the present application is directed to a method for preparing a compound of formula XIX, or a salt thereof, comprising converting any of the compounds of formulae XX-XXI to the compound of formula XIX using the methods described herein.

In another embodiment, the present application is directed to a method for preparing a compound of formula XX, or a salt thereof, comprising converting any of the compounds of formula XXI to the compound of formula XX using the methods described herein.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

Another embodiment of the present invention is directed to compounds of the following formula

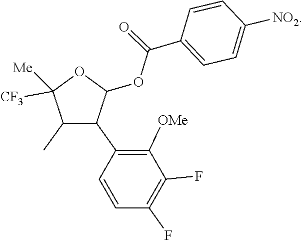 [SMILES: COc1c(C2C(OC(=O)c3ccc([N+](=O)[O-])cc3)OC(C)(C(F)(F)F)C2C)ccc(F)c1F]

Another embodiment of the present invention is directed to compounds of the following formula

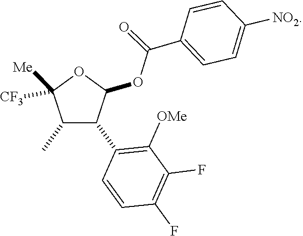

Another embodiment of the present invention is directed to a compound of the following formula

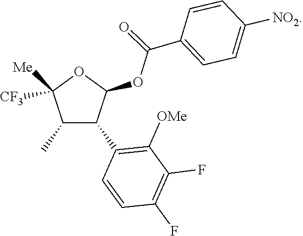 [SMILES: COc1c([C@H]2[C@H](OC(=O)c3ccc([N+](=O)[O-])cc3)O[C@@](C)(C(F)(F)F)[C@H]2C)ccc(F)c1F]

Another embodiment of the present invention is directed to compounds of the following formulae:

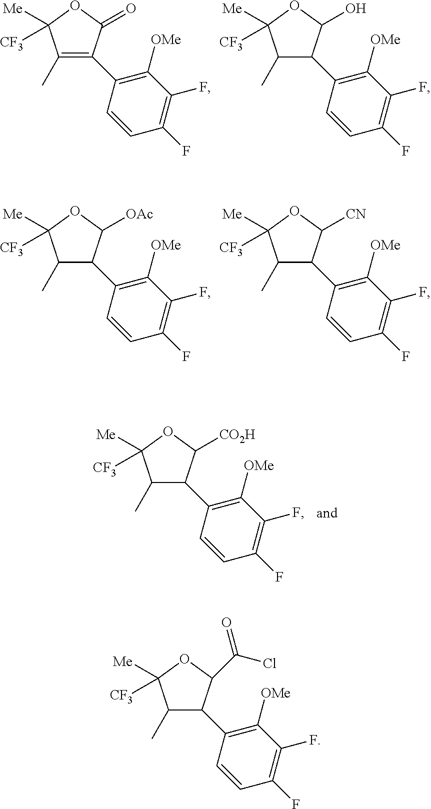 [SMILES: COc1c(C2=C(C)C(C)(C(F)(F)F)OC2=O)ccc(F)c1F ; COc1c(C2C(O)OC(C)(C(F)(F)F)C2C)ccc(F)c1F ; COc1c(C2C(OC(C)=O)OC(C)(C(F)(F)F)C2C)ccc(F)c1F ; COc1c(C2C(C#N)OC(C)(C(F)(F)F)C2C)ccc(F)c1F ; COc1c(C2C(C(=O)O)OC(C)(C(F)(F)F)C2C)ccc(F)c1F ; COc1c(C2C(C(=O)Cl)OC(C)(C(F)(F)F)C2C)ccc(F)c1F]

Another embodiment of the present invention is directed to compounds of the following formulae:

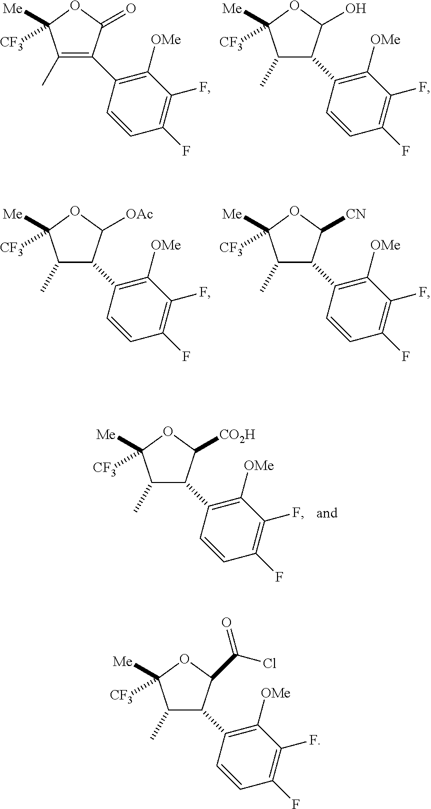

Another embodiment of the present invention is directed to compounds of the following formulae:

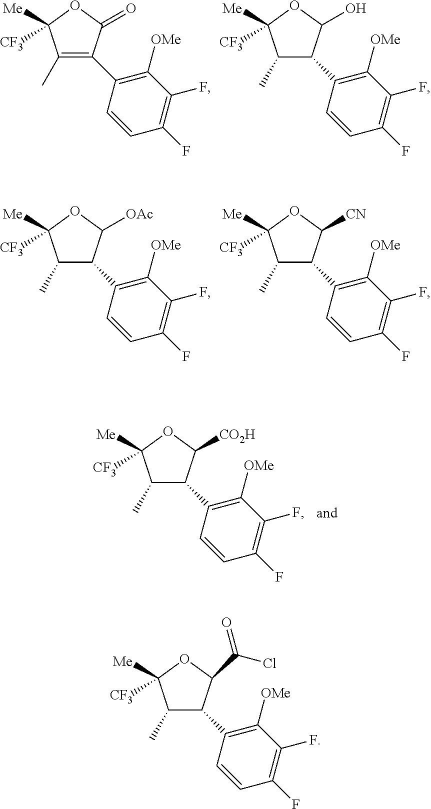 [SMILES: COc1c(C2=C(C)[C@](C)(C(F)(F)F)OC2=O)ccc(F)c1F ; COc1c([C@H]2C(O)O[C@@](C)(C(F)(F)F)[C@H]2C)ccc(F)c1F ; COc1c([C@H]2C(OC(C)=O)O[C@@](C)(C(F)(F)F)[C@H]2C)ccc(F)c1F ; COc1c([C@H]2[C@H](C#N)O[C@@](C)(C(F)(F)F)[C@H]2C)ccc(F)c1F ; COc1c([C@H]2[C@H](C(=O)O)O[C@@](C)(C(F)(F)F)[C@H]2C)ccc(F)c1F ; COc1c([C@H]2[C@H](C(=O)Cl)O[C@@](C)(C(F)(F)F)[C@H]2C)ccc(F)c1F]

Another embodiment of the present invention is directed to compounds of the following formulae:

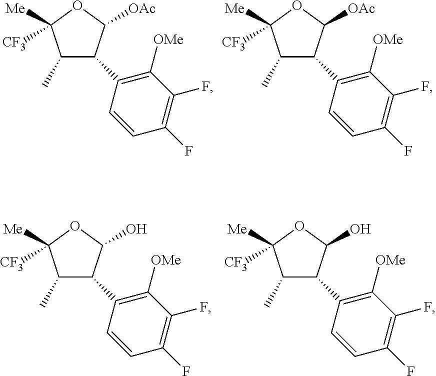 [SMILES: COc1c([C@H]2[C@@H](OC(C)=O)O[C@@](C)(C(F)(F)F)[C@H]2C)ccc(F)c1F ; COc1c([C@H]2[C@H](OC(C)=O)O[C@@](C)(C(F)(F)F)[C@H]2C)ccc(F)c1F ; COc1c([C@H]2[C@@H](O)O[C@@](C)(C(F)(F)F)[C@H]2C)ccc(F)c1F ; COc1c([C@H]2[C@H](O)O[C@@](C)(C(F)(F)F)[C@H]2C)ccc(F)c1F]

Another embodiment of the present invention is directed to compound salts of the following formulae:

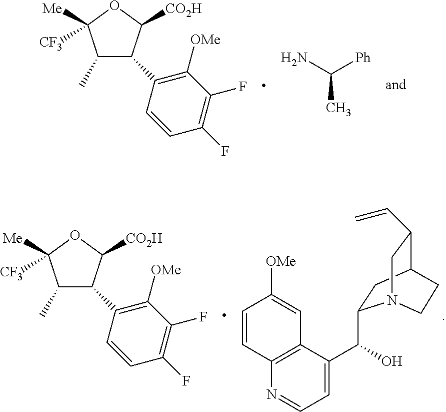

As used herein, in any chemical structure or formula, a bold or hashed straight bond ( [symbol] or [symbol] , respectively) attached to a stereocenter of a compound, such as in

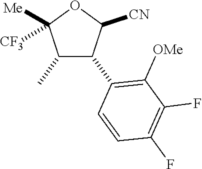 [SMILES: COc1c([C@H]2[C@H](C#N)O[C@@](C)(C(F)(F)F)[C@H]2C)ccc(F)c1F]

denotes the relative stereochemistry of the stereocenter, relative to other stereocenter(s) to which bold or hashed straight bonds are attached.

As used herein, in any chemical structure or formula, a bold or hashed wedge bond ( [symbol] or [symbol] , respectively) attached to a stereocenter of a compound, such as in

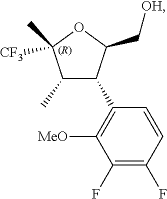

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and (S)-designators in the chemical name reflect the relative stereochemistry of the compound.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound.

As used herein, the term “compound,” when referring to the compounds described in this application, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance concentration of hydrogen.

As used herein, “¹H” refers to protium. Where an atom in a compound of the invention, or a pharmaceutically acceptable salt thereof, is designated as protium, protium is present at the specified position at at least the natural abundance concentration of protium.

As used herein, “D,” “d,” and “²H” refer to deuterium.

In some embodiments, the compounds described in the present application include each constituent atom at approximately the natural abundance isotopic composition of the specified element.

In some embodiments, the compounds described in the present application, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O ¹⁷O, and ³¹P, respectively. The term the “compound of formula” followed by a number (typically Roman number) and the term “compound” followed by the same number (Roman or otherwise) may interchangeably be used. For example, the “compound of formula V” and “compound V” denote the same compound.

The term “reacting,” when referring to a chemical reaction, means to add or mix two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “conducted in a solvent,” when referring to a reaction, means that the substrate(s) and reagent(s) are dissolved or suspended in the specified solvent or in a mixture of solvents comprising the specified solvent.

The term “chromatographic purification” refers to any method of purification based on differential retention by a stationary phase. Methods of chromatographic purification include flash chromatography, medium pressure liquid chromatography, preparative thin layer chromatography, and high performance liquid chromatography.

The term “converting,” as used herein to refer to a step of converting a first compound or salt to a second compound or salt, refers to a process of transforming the first compound or salt to the second compound or salt in one or more chemical steps.

The term “acid” refers to a chemical species having a pKa (in water) of less than 7. The term includes inorganic (mineral) acids, such as hydrochloric acid, hydrobromic acid, hydroiodic acid, sulfuric acid, phosphoric acid, nitric acid, and the like. The term also includes organic acids such as acetic acid, propionic acid, n-butyric acid, i-butyric acid, n-valeric acid, i-valeric acid, n-hexanoic acid, succinic acid, glutaric acid, adipic acid, aspartic acid, formic acid, citric acid, o-chlorobenzoic acid, chloroacetic acid, dichloroacetic acid, trichloroacetic acid, nicotinic acid, lactic acid, oxalic acid, picric acid, picolinic acid, fluoroacetic acid, difluoroacetic acid, trifluoroacetic acid, phthalic acid, isophthalic acid, terephthalic acid, maleic acid, malonic acid, and the like.

The term “base” refers to a chemical species whose conjugate acid has a pKa (in water) of greater than 7. The term includes “inorganic bases,” such as sodium hydroxide, potassium hydroxide, sodium bicarbonate, potassium bicarbonate, sodium carbonate, potassium carbonate, cesium carbonate, potassium phosphate (mono-, di-, or tri-basic), sodium hydride, and potassium hydride. The term also includes “anionic organic bases,” such as methyl lithium, butyl lithium, lithium diisopropyl amide, and sodium acetate. The term also includes “neutral organic bases,” such as trimethylamine, dimethylethylamine, diethylmethylamine, triethylamine, di-n-propylmethylamine, dimethylcyclohexylamine, diisopropylethylamine, tri-n-propylamine, diisopropylisobutylamine, dimethyl-n-nonylamine, tri-n-butylamine, di-n-hexylmethylamine, dimethyl-n-dodecylamine, tri-n-pentylamine, 1,4-diazabicyclo[2.2.2]octane (DABCO), dimethylaminopyridine (DMAP), 1,5-diazabicyclo[4.3.0]non-5-ene (DBN), 1,8-diazabicyclo[5.4.0]undec-7-ene (DBU), pyridine, 2,3-lutidine, 2,4-lutidine, 2,5-lutidine, 2,6-lutidine, 3,4-lutidine, 3,5-lutidine, 2,3,4-collidine, 2,4,5-collidine, 2,5,6-collidine, 2,4,6-collidine, 3,4,5-collidine, and 3,5,6-collidine.

The term “alcohol protecting group” refers to a chemical moiety suitable to protect an alcohol group against undesirable side reactions during synthetic procedures. Common alcohol protecting groups include methyl, ethyl, isopropyl, benzyl, 2-tetrahydropyranyl, acetyl, trifluoroacetyl, trialkylsilyl, aryldialkylsilyl, alkyldiarylsilyl, or triarylsilyl. Other alcohol protecting groups also are well known in the art. See, e.g., P. G. M. Wuts et al., Greene's Protective Groups in Organic Synthesis (4th ed. 2006).

The term “deprotecting” refers to a step of reacting a compound or salt containing a protecting group, such as an alcohol protecting group, under conditions suitable to remove the protecting group and reveal the protected moiety. For example, where a compound or salt contains an alcohol protecting group, the term “deprotecting” refers to reacting the compound or salt under conditions suitable to remove the alcohol protecting group and reveal the alcohol. Conditions for removing various protecting groups are well known in the art. See, e.g., P. G. M. Wuts et al., Greene's Protective Groups in Organic Synthesis (4th ed. 2006).

The term “hydrogenation catalyst” refers to any homogeneous or heterogeneous catalyst that catalyzes the hydrogenolysis of benzylic carbon-oxygen single bonds. Suitable hydrogenation catalysts are well-known in the art and include palladium on activated carbon, platinum oxide, and Raney Nickel.

The term “coupling,” when referring to a reaction between a carboxylic acid or acid halide and an amine, refers to a net transformation linking the carboxylic acid or acid halide and the amine to form an amide. The term includes a direct reaction between the carboxylic acid and the amine, as well as a reaction between an activated derivative of the carboxylic acid (such as the derivative formed by the reaction between the carboxylic acid and a coupling reagent) and the amine.

The term “coupling reagent” refers to a reagent suitable to react with a carboxylic acid to activate the carboxylic acid for coupling with an amine to form an amide bond. Coupling reagents are well known in the art. Coupling reagents include, but are not limited to, thionyl chloride, oxalyl chloride, 1,1′-carbonylbis-(4,5-dicyanoimidazole) (CBDCI), 1,1′-carbonyldiimidazole (CDI), propylphosphonic anhydride (T3P), 1-ethyl-3-(3-dimethylaminopropyl)carbodiimide (EDCI), N, N′-dicyclohexylcarbodiimide (DCC), 1-[Bis(dimethylamino)methylene]-1H-1,2,3-triazolo[4,5-b]pyridinium 3-oxid hexafluorophosphate (HATU), and 1-hydroxybenzotriazole (HOBt).

The term “monovalent cation” refers to any cation with a charge of +1, such as alkali metal cations, NH₄⁺, and tetraalkylammonium.

The term “alkali metal cation” refers to a cation derived from a Group I metal atom, including without limitation lithium (Li⁺), sodium (Na⁺), potassium (K⁺), rubidium (Rb⁺), and cesium (Cs⁺).

The term “substituted benzyl” refers to a benzyl group that is substituted with 1-3 substituents selected from the group consisting of C₁-C₃ alkyl, C₁-C₃ alkoxy, halogen, and cyano.

The term “ketone solvent” refers to a compound having the formula CₙH₂ₙ₊₁ C(O)CₘH₂ₘ₊₁, wherein n and m are each independently an integer between 1 and 6. The CₙH₂ₙ₊₁ and CₘH₂ₘ₊₁ and groups may be linear or branched and each may be substituted with up to 3 halogens. Ketone solvents include without limitation acetone, methyl ethyl ketone, 3-pentanone, and methyl tert-butyl ketone.

The term “ethereal solvent” refers to an organic solvent having at least one ether moiety. Ethereal solvents include without limitation tetrahydofuran, dimethoxyethane, dioxane, and dialkyl ethers such as diethyl ether and methyl isobutyl ether.

The term “ester solvent” refers to a compound having the formula CₙH₂ₙ₊₁OC(O)CₘH₂ₘ₊₁, wherein n and m are each independently an integer between 1 and 6. The CₙH₂ₙ₊₁ and CₘH₂ₘ₊₁ and groups may be linear or branched and each may be substituted with up to 3 halogens. Ester solvents include without limitation ethyl acetate, isopropyl acetate, butyl acetate, and ethylpropionate.

The term “halogenated solvent” refers to a C₁-C₆ alkane or C₂-C₆ alkene substituted with up to six halogens. Halogenated solvents include without limitation dichloromethane, dichloroethane, chloroform, tetrachloroethylene, and carbon tetrachloride.

The term “aromatic solvent” refers to a C₆₋₁₀ aromatic hydrocarbon. The aromatic hydrocarbon may be substituted with up to six halogens. Aromatic solvents include without limitation benzene, trifluoromethylbenzene, xylene, and toluene.

The term “about” means that the stated number can vary from that value by ±10%. Where the term defines a temperature, the stated temperature can vary by ±10%. For example, about 80° C. means between 72° C. and 88° C. Where the term defines pressure, the term “about” means the pressure can vary by ±10%. Thus, about 100 bars means between 90 and 110 bars. Where the term defines quantity (such as equivalents or weight), the term means the quantity can vary by ±10%. For example, about 1 equivalent means between 0.9 and 1.1 equivalents. Where the term defines time, the term means the stated time can vary by ±10%. For example, about 1 hour means between 0.9 and 1.1 hours.

The term “leaving group” is a chemical group that is readily displaced by a desired incoming chemical moiety. Thus, the choice of the specific suitable leaving group is predicated upon its ability to be readily displaced by the incoming chemical moiety such as a CN group. Suitable leaving groups are well known in the art, e.g., see, “Advanced Organic Chemistry,” Jerry March, 5.sup.th Ed., pp. 351-357, John Wiley and Sons, N.Y. For the purposes of converting compound IX to compound VII, the leaving group on compound VIII is any group of formula OC(═O)—Z, OC(═O)OZ, OC(═O)CH═CH—Z, or OP(═O)Z₂ wherein Z may be an unsubstituted aryl or an aryl substituted by CN, halo, NO₂, or a short chain alkyl, alkoxy, haloalkyl, or haloalkoxy group wherein the short chain comprises 1, 2, 3, or 4 carbon atoms. Alternatively, Z is a short chain (i.e., with 1-4 carbon atoms) alkyl or haloalkyl group. Examples of aryl groups include phenyl and naphthyl.

As used here, the term “cyanating agent” (such as trimethylsilyl cyanide, diethylaluminum cyanide, KCN, NaCN, TBACN, HCN and the like) to afford the compound of formula VIII. In one embodiment, the reaction between the cyanating agent (e.g., trimethylsilyl cyanide) and the compound of formula VIII may be conducted in the presence of a Lewis acid. In some embodiments, the Lewis acid is boron trifluoride ethyl etherate (BF₃OEt₂), TiCl₄, InCl₃, AgSbF₆, iodine, ZnBr₂, Al(OiPr)₃, MgCl₂, Mn(acac)₂, MnCl₂, TMSOTf, SnCl₄, and the like. In further embodiments, the Lewis acid is BF₃OEt₂. The cyanation reaction may be conducted in an organic solvent, for example toluene, dichloromethane, 2-methyl THF, acetonitrile, methanol, 1,2-dichloroethane, nitromethane, and the like.

### Uses of Compounds and Pharmaceutically Acceptable Salts and Compositions

In another aspect, the invention features a method of inhibiting a voltage-gated sodium channel in a subject comprising administering to the subject a compound of formula I or a pharmaceutically acceptable salt thereof or a pharmaceutical composition thereof.

In one aspect, the disclosure relates to a method of treating or lessening the severity of pain in a subject, comprising administering to the subject a compound of formula I, or a pharmaceutically acceptable salt thereof.

In another aspect, the disclosure relates to a use of a compound of formula I, or a pharmaceutically acceptable salt thereof, in a method of treating or lessening the severity of pain in a subject, comprising administering to the subject a compound of formula I, or the pharmaceutically acceptable salt thereof.

In another aspect, the disclosure relates to a composition comprising a compound of formula I, or a pharmaceutically acceptable salt thereof, for use in a method of treating or lessening the severity of pain in a subject, wherein the composition is prepared for administration of a compound of formula I, or the pharmaceutically acceptable salt thereof, to the subject.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P, ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)—and/or carbon—14 (¹⁴C)—labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### EXAMPLES

General methods. ¹H NMR (400 MHz) spectra were obtained as solutions in an appropriate deuterated solvent such as dimethyl sulfoxide-d₆ (DMSO-d₆).

Analytical supercritical fluid chromatography (SFC) separation of various isomeric mixtures was accomplished using a Waters UPC2-SFC instrument comprising a convergence manager, a sample manager, a binary solvent manager, a column manager-30S, a PDA detector, an isocratic solvent manager and a QDa detector. Columns used include those by manufactured by Regis Technologies (e.g., R′R Whelk 0-1, 3.5 μm particle size, 5.0 cm×3.0 mm size) with a mobile phase of Solvent A: liquid CO₂ (58-60 bar/40° C.) Solvent B: methanol HPLC grade with 20 mM NH₃ at a flow rate of 2 ml/min and an injection volume of 2 μl. Gradient: at 0 min (95:5) A:B, at 3.5 min (50:50) A:B, at 3.55 min (40:60) A:B, at 3.95 min (40:60) A:B and at 4.0 min (95:5) A:B. Samples for analytical SFC were dissolved in methanol at approximately 0.5 mg/ml concentration.

Preparative SFC used the same stationary and mobile phases as those described herein for analytical SFC but the samples were purified using a different instrument and gradient method as follows. Preparative SFC separation of various isomeric mixtures was accomplished using a Waters Prep-100 SFC instrument comprising a Back Pressure Regulator, a 2767 Sample Manager, a 2545 Quarternary Gradient Module, a Column Oven, a 2998 PDA detector, an Isocratic Solvent Manager, a P-200 CO₂ pump, SFC Flow Splitter-100, 3 Heat exchangers, a Series III LC pump and a QDa detector. Columns used include those manufactured by Regis Technologies (e.g., R′R Whelk 0-1, 5.0 μm particle size, 25.0 cm×21.1 mm size) with a mobile phase of Solvent A: liquid CO₂ (58-60 bar/40° C.) Solvent B: methanol HPLC grade with 20 mM NH₃ at a flow rate of 100 ml/min and an injection volume of 500 μl (50 mg crude loading), 2:1 ratio of methanol to dichloroethane was used for solubilization and SFC injection of crude compound. For injection 500 μl/50 mg loading the following method was used: Isocratic: at 0 min to 7.6 min (80:20) A:B, Gradient: at 8.1 min (75:25) A:B, Isocratic at 8.2 to 10.6 min (75:25) (A:B), Gradient: at 10.7 min (80:20) A:B and Isocratic: at 11 min (80:20) (A:B). For injection 1500 μl/150 mg loading the following method was used: Isocratic: at 0 min to 7.5 min (80:20) A:B, Gradient: at 7.6 min (75:25) A:B, Gradient: at 8.1 min (60:40) A:B, Isocratic: at 8.7 min to 10.6 min (60:40) A:B, Gradient: at 10.7 min (80:20) A:B and Isocratic: at 12 min (80:20) A:B.

LC/MS Method: LC/MS analysis was conducted using an Acquity UPLC BEH C₈ column (50×2.1 mm, 1.7 μm particle) made by Waters (pn: 186002877) with a (2.1×5 mm, 1.7 μm particle) guard column (pn: 186003978), and a dual gradient run from 2-98% mobile phase B over 4.45 minutes. Mobile phase A=H₂O (10 mM ammonium formate with 0.05% ammonium hydroxide). Mobile phase B=acetonitrile. Flow rate=0.6 mL/min, injection volume=2 μL, and column temperature=45° C.

Solid state NMR analysis was conducted on a Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

Thermogravimetric analysis (TGA) data were collected on a TA Discovery Thermogravimetric Analyzer or equivalent instrumentation. A sample with weight of approximately 1-5 mg was scanned from 25° C. to 350° C. at a heating rate of 10° C./min. Data were collected by Thermal Advantage Q Series™ software and analyzed by Trios and/or Universal Analysis software (TA Instruments, New Castle, DE).

Differential scanning calorimetry (DSC) data were acquired using a TA Instruments Q2000 or equivalent instrumentation. A sample with a weight between 1 and 10 mg was weighed into an aluminum pan. This pan was placed in the sample position in the calorimeter cell. An empty pan was placed in the reference position. The calorimeter cell was closed and a flow of N₂ gas was passed through the cell. The heating program was set to heat the sample at a heating rate of 10° C./min to a temperature of 300° C. When the run was completed, the data were analyzed by Trios and/or Universal Analysis software (TA Instruments, New Castle, DE).

Infrared (IR) spectra were collected using a Thermo Scientific Nicolet iS50 Spectrometer equipped with a diamond ATR sampling accessory.

### Abbreviations

Unless otherwise noted, or where the context dictates otherwise, the following abbreviations shall be understood to have the following meanings:

| Abbreviation | Meaning |
|---|---|
| NMR | Nuclear magnetic resonance |
| LC/MS | Liquid chromatography-mass spectrometry |
| UPLC | Ultra performance liquid chromatography |
| HPLC/MS/MS | High performance liquid chromatography/tandem |
| | mass spectrometry |
| IS | Internal standard |
| HPLC | High performance liquid chromatography |
| SFC | Supercritical fluid chromatography |
| MDAP | Mass directed auto purification |
| g | grams |
| mg | milligrams |
| L | Liter(s) |
| mL | Milliliters |
| μL | Microliters |
| mmol | millimole |
| h | hours |
| min | Minutes |
| MHz | Megahertz |
| Hz | Hertz |
| N | Normal (concentration) |
| M | Molar (concentration) |
| mM | Millimolar (concentration) |
| tBuOH | tert-butyl alcohol |
| nBuAc | n-butyl acetate |
| CPME | Cyclopentyl methyl ether |
| DCM | Dichloromethane |
| DMF | N,N-Dimethylformamide |
| DMSO | Dimethyl sulfoxide |
| DRG | Dorsal root ganglia |
| EtOH | Ethanol |
| EtOAc | Ethyl acetate |
| HATU | 1-[Bis(dimethylamino)methylene]-1H-1,2,3- |
| | triazolo[4,5-b]pyridinium 3-oxide |
| | hexafluorophosphate |
| EDCI | 1-Ethyl-3-(3-dimethylaminopropyl)carbodiimide |
| IPA | Isopropyl alcohol |
| T3P | Propylphosphonic anhydride, i.e., 2,4,6-tripropyl- |
| | 1,3,5,2,4,6-trioxatriphosphinane 2,4,6-trioxide |
| MeOH | Methanol |
| MTBE | Methyl tert-butyl ether |
| MSA | methane sulfonic acid |
| THF | Tetrahydrofuran |
| Mn(acac)₂ | manganese(II) acetylacetonate |
| TEA | triethylamine |
| RB(F) | Round bottom (flask) |
| RT | Room temperature |
| TFT | α,α,α-Trifluorotoluene |
| TMSOTf | trimethylsilyl trifluoromethanesulfonate |
| DIPEA | N,N-diisopropylethyl amine |
| NMM | N-methylmorpholine |
| TBD | Triazabicyclodecene |
| DBU | 1,8-diazabicyclo[5.4.0]undec-7-ene |
| DABCO | 1,4-diazabicyclo[2.2.2] octane |
| MTBD | 7-Methyl-1,5,7-triazabicyclo [4.4.0]dec-5-ene |
| LiHMDS | Lithium hexamethyldisilazide (LiN(SiMe₃)₂) |

### Example 1

### Synthesis of 4-((2R,3S,4S,5R)-3-(3,4-difluoro-2-methoxyphenyl)-4,5-dimethyl-5-(trifluoromethyl)tetrahydrofuran-2-carboxamido)picolinamide (I)

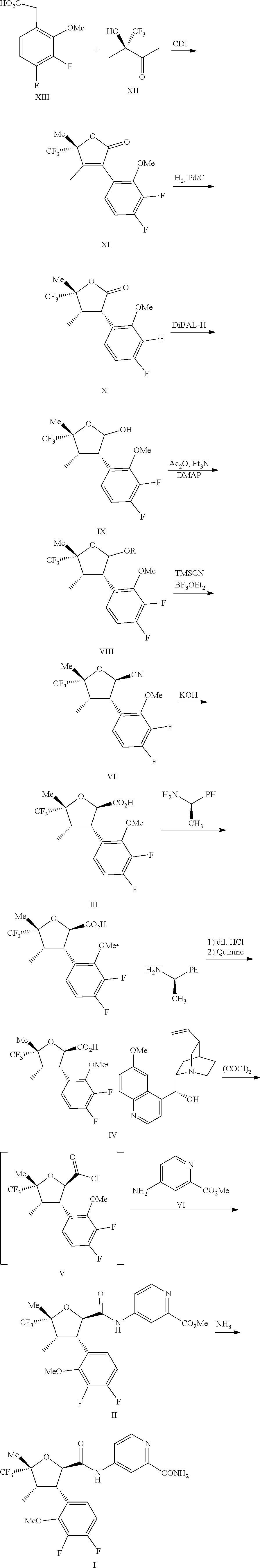

### Step 1

### Synthesis of (R)-3-(3,4-difluoro-2-methoxyphenyl)-4,5-dimethyl-5-(trifluoromethyl)furan-2(5H)-one (XI)

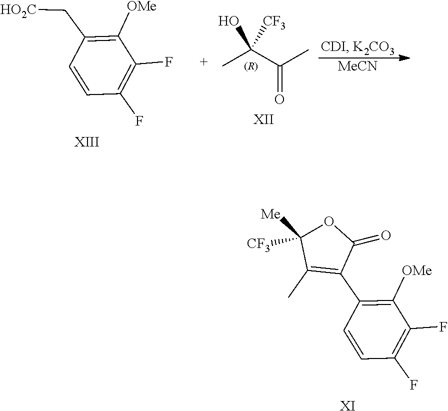

Compound XIII (Alichem, 3.832 kg, 18.955 mols) was added to a 140 L reactor that had been previously vacuum-dried and flushed with N₂ gas. Anhydrous acetonitrile, 18.85 L, was added, and the solution was cooled to −2° C. Carbonyldiimidazole (Chem Impex, 99.5%, 3.280 kg, 19.92 mols, 1.05 eq) was added in portions (from 4×820 g bottles, preweighed in a N₂ drybox. Each bottle of CDI was added 1-2 minutes apart to avoid a sudden gas evolution of CO₂. The temperature went to +2° C. during the addition. The solution was stirred at 0° to −2° C. for 1.5 hours. A solution of XII in THF (containing 4.028 kg of XII; 20.42 mols, 1.077 eq) was added fast with a metering pump. The pump and container were rinsed with 2 L of anhydrous acetonitrile, potassium carbonate, 325 mesh anhydrous (3.276 kg, 23.70 mols, 1.25 eq) was quickly added, and the reaction mixture was stirred at 35° C. for 5 hours, then cooled to 15° C. overnight. MTBE (24.5 L) was added followed by 62.4 kg of 0.62N H₂SO₄ and then a 5 L rinse of DI water. The aqueous layer (pH 8) was re-extracted with 20 L of MTBE. The total MTBE was vacuum concentrated to a dry solid, and re-concentrated with 10 L IPA to a dry solid.

For recrystallization, the solid was dissolved in 25.5 L of IPA and transferred to the 140 L reactor with a 5 L rinse of IPA; the solution was warmed to 35° C. A pump was set up for delivery of 47.1 kg of DI water; this was added to the IPA solution slowly over 2.5 hours. After stirring the slurry of crystals for another 2 hours, the slurry was ramped down over approx. 3 hours to 15° C. and continued stirring for another 12 hours. The slurry was filtered, washed with 2×6 L of 1:4 IPA:DI water, and dried with single pass heated N₂ gas (N₂ gas was heated to 75° C., the cake temperature was ca 50° C.) for 3 days to constant weight. Final weight of product was 5.205 kg, 85.2%. The product gave a proton NMR spectrum consistent with the structure of XI.

### Step 2

### Synthesis of (3S,4S,5R)-3-(3,4-difluoro-2-methoxyphenyl)-4,5-dimethyl-5-(trifluoromethyl)dihydrofuran-2(3H)-one (X)

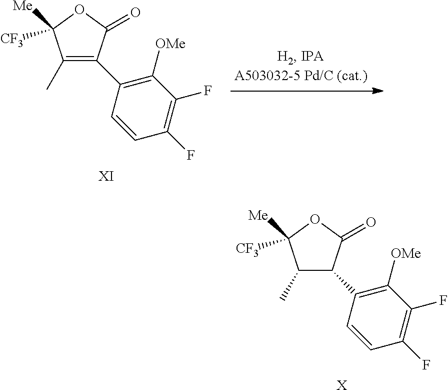

### Procedure 1:

Compound XI obtained from the reaction above (1200 g, 3.72 mol) was added to the 11 L hydrogenation reactor (previously flushed with N₂ gas and set for a slow N₂ gas sweep). The catalyst (1200 g, 4.89% Pd, 63.8% water, corresponding to 21.2 g Pd, 0.0536 molar eq.) was added. Isopropanol (7.0 L) was added. The reactor was sealed, purged 3×N₂ gas/vacuum, then with 50 psi nitrogen/hydrogen, and finally adjusted to 225 psi hydrogen. The jacket was set to 30-31° C. and agitation was started. Agitation was continued for 30 hours; an NMR sample (0.2 ml+2.0 ml MTBE+ 1.0 ml of 5% KHCO₃; evaporation of 1.5 ml of the MTBE, and NMR); showed 2.8% starting material. Reaction was considered complete. The slurry was filtered (solka-floc) and washed with 10 L of IPA, then 3 L DCM. The filtrate was concentrated to an oil, and redissolved in 3 L of toluene. The toluene solution was re-concentrated to an oil, 1152 g, 3.553 mols, 95.5% yield. Proton NMR of the product was consistent with compound X.

### Procedure 2:

To a reactor rated for hydrogen and pressure service, and equipped with a gas dosing unit and pressure controller, was charged Compound XI (1 equiv, limiting reagent), 5% palladium on carbon (0.05 equiv, corrected for water content and palladium assay), tetrahydrofuran (1.75 volumes), 2-propanol (5.25 volumes), and trifluoroacetic acid (0.05 equiv). The vessel was pressurized to 3 barg with nitrogen, and then vented to ambient pressure. This sequence was performed 3 times. The reactor contents were adjusted to 30° C. The vessel was then pressurized to 3 barg with hydrogen, and vented to ambient pressure. This sequence was performed 3 times. The reactor was then pressurized to operating pressure (40 barg) with hydrogen, and agitation was started at a sufficient speed to achieve gassing of the liquid from the head space.

The reaction mixture was stirred at these conditions until reaction completion (Less than 1% Compound XI and its diastereomers by GC).

The hydrogen in the headspace was vented. The reactor was pressurized to 3 barg with nitrogen and the reactor was vented. This sequence was performed 3 times. Cyclohexene (0.2 volumes) was charged to the reactor, and the reaction was maintained at 30° C. with stirring under nitrogen for no less than 15 minutes.

The reaction mixture was filtered over a bed of diatomaceous earth to remove the catalyst. The filter cake was washed with 2-propanol (4 volumes). The filtrates from the primary filtration and the wash were combined.

In a well mixed vessel, the filtrate was concentrated under reduced pressure at no more than 40° C. to a total of 3 volumes. Toluene (7 volumes) was charged and distillation was resumed under reduced pressure at no more than 50° C. until reaching a total of 3 volumes. Toluene (7 volumes) was charged and distillation was resumed under reduced pressure at no more than 50° C. until reaching a total of 3 volumes. Toluene (5 volumes) was charged and the solution was mixed well. Proton NMR of the product was consistent with compound X.

¹H-NMR CDCl₃: δ 6.93-6.80 (m, 2H); 4.48 (d, 1H, J=9.5 Hz); 4.03 (d, 3H, J=3.1 Hz); 2.89 (dq, 1H, J=9.5, 7.5 Hz); 1.71 (d, 3H, J=1.2 Hz); 0.84-0.76 (m, 3H) ppm.

### Step 3

### Synthesis of (2S,3S,4S,5R)-3-(3,4-difluoro-2-methoxyphenyl)-4,5-dimethyl-5-(trifluoromethyl)tetrahydrofuran-2-ol (IX)

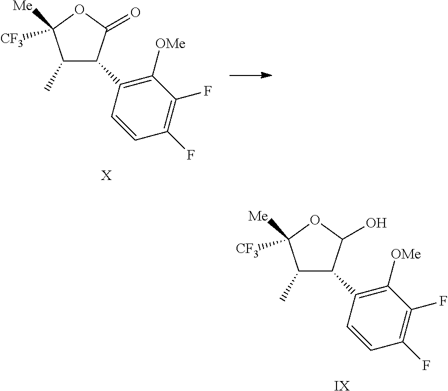

### Procedure 1:

The product of the reaction above (compound X, 1052 g, 3.2445 mols) was added to a 50 L jacketed reactor, and added 8.82 L of anhydrous toluene under N₂ gas. The resulting solution was cooled to −31° C. overnight under N₂ gas. Diisobutylaluminum hydride (1.96 kg of 25% solution in toluene, 3.445 mol, 1.056 eq) was slowly transferred to the reaction vessel through an addition funnel under N₂ gas. The hydride reagent was added to the reaction solution over 2 hours and the reactor temperature went from −31.6° to −27.4° C. during the addition. The solution was stirred at −26 to −27° C. for 90 minutes. A solution of 2.75 kg of potassium/sodium tartrate in 20 L of DI water was added over 2.5 hours. The reaction mixture was allowed to warm up until it reached 0° C., the cooler was turned to +24° C. after about 2 hours of addition. Toluene (5 L) was added and the mixture was stirred overnight at +20° C.

The reaction mixture was transferred to a separatory funnel and separated aqueous phase from the organic phase. The aqueous phase was re-extracted with 5 L of toluene. The two toluene solutions were combined, treated with magnesium sulfate, and filtered. The solid was washed with toluene, and the combined toluene solution was vacuum concentrated to an oil, wt. 1055 g, 3.2336 mol, 99.7% of crude lactol. The crude product was directly taken into the next step (acetylation is exemplified below). The proton NMR of the crude lactol was consistent with compound IX.

### Procedure 2:

A solution of Compound X in toluene (targeting 8 vol) was added to a reactor. Agitation was begun and mixture was cooled to −25±5° C. A solution of diisobutylaluminum hydride (25% w/w toluene) was added to the reaction mixture while maintaining the batch temperature at no more than −20° C. Temperature was adjusted to −25±5° C. and the batch was stirred for no less than one hour. Upon reaction completion, a solution of acetone in toluene (0.3 equiv in 0.5 vol) was added to the reaction mixture, maintaining temperature at −25±5° C. and stirred for no less than 30 minutes. Reaction mixture was then warmed to 0±5° C. A 12 vol solution of 0.62 M citric acid was transfered to the reactor while maintaining the temperature at 20±5° C. and the biphasic mixture was stirred for no less than four hours. Phases were allowed to settle and the bottom, aqueous phase was drained off. A 12 vol solution of 0.62 M citric acid was added to the batch and the biphasic mixture was stirred at 20±5° C. for no less than 30 minutes. Phases were allowed to settle and the bottom, aqueous layer was drained off. 5 vol of water was charged to the batch and the biphasic mixture was stirred at 20±5° C. for no less than 30 minutes. Phases were allowed to settle and the bottom, aqueous layer was drained off. The organic layer was distilled to a total of 5 vol while maintaining an internal temperature at or below 45° C. 5 vol of toluene was charged and the mixture was distilled to a total of 5 vol. Distillation continued until residual water was below 0.1%. The proton NMR of the crude lactol was consistent with compound IX.

¹H-NMR CDCl₃: δ 7.30-7.26 (m, 1H); 7.20-7.18 (m, 1H); 5.81 (d, 1H, J=4 Hz); 4.00 (s, 3H); 3.84-3.80 (m, 1H); 2.92-2.88 (m, 1H); 1.67 (s, 3H); 0.83 (d, 3H, J=8 Hz) ppm

### Step 4

### Synthesis of (3S,4S,5R)-3-(3,4-difluoro-2-methoxyphenyl)-4,5-dimethyl-5-(trifluoromethyl)tetrahydrofuran-2-yl acetate (VIII)

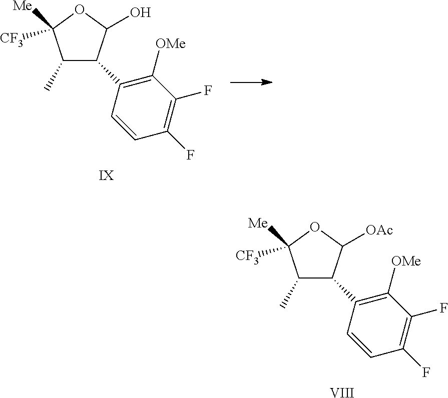

The crude product of the reaction above (1055 g of compound IX, 3.23 mols) was transferred to a 25 L jacketed reactor predried and kept under N₂ gas. Crude compound IX was rinsed in with anhydrous toluene, 6.8 L and the mixture stirred to ensure complete solution at 20° C. Triethylamine (466 ml, 3.343 mols, 1.04 eq) was added, followed by DMAP (3.58 g, 0.0293 mols, 0.01 eq). Acetic anhydride, (313 ml, 3.288 mols, 1.02 eq) was added over about 5-10 minutes using an addition funnel (T rose from 21.1° C. to a T=26.4° C. during addition of acetic anhydride). The reaction mixture was stirred at 25° C. for 100 minutes. Proton NMR of the reaction mixture indicated that the reaction was complete at this point.

The reaction mixture was extracted with 3.5 L of 25% ammonium chloride, then 1200 ml of 10% KHCO₃. The aqueous layers were re-extracted with 2 L of toluene and the combined toluene solution was dried over magnesium sulfate, filtered and washed the filtered solid with toluene. The resulting toluene solution was concentrated to dryness, initially with a 20 L RB flask and then using a 3 L jacketed reactor (1898 g transferred) for the final concentration. The solution was vacuum-distilling to a thick oil, placed it on high-vacuum at 25° C. with stirring until the oil converts to a crystalline mass (with a small amount of a viscous oil remaining). Hexane (900 ml) was added to the reaction vessel (containing solid and some remnant oil) and stirred the mixture at 20° C. overnight. The mixture was cooled to 10° C. and stirred for 2 hours, then cooled to 4° C. while stirring for 6 hours, before cooling the mixture to −10° C. overnight (while stirring), and finally to −14° C. over the weekend (36 to 48 hours) to obtain a crystal slurry. The slurry was filtered with a jacketed filter at −15° C., and then washed the solid with cold (−16° C.) hexane (2×150 ml, then 100 ml). The crystals were dried at RT under vacuum. The resulting solid was dissolved in toluene (total 1450 ml solution) and assayed to obtain 800.2 g in solution.

Additional product (that was stuck to the reactor) was dissolved off with toluene to give 425 ml of solution; assay gave 188.4 g product. The mother liquors were concentrated to 117 g of an oil. Proton NMR showed about 55% product, and 45% impurities.

The total yield (crystals filtered from reaction mixture and solids dissolved from the reactor surface) provided 988.6 g, 2.68 mols, 83.1% of compound VIII (acetate ester).

The product (988.6 g compound VIII-acetate ester) was used in the next reaction without any further purification.

### Synthesis of (2S,3S,4S,5R)-3-(3,4-difluoro-2-methoxyphenyl)-4,5-dimethyl-5-(trifluoromethyl)tetrahydrofuran-2-yl4-nitrobenzoate (VIII, 4-nitrobenzoate ester)

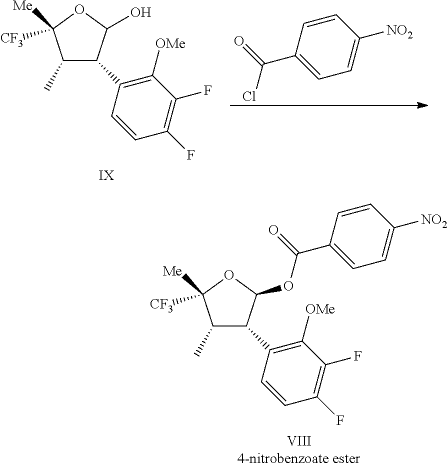

Compound IX in a solution of 5 vol toluene was charged to the reactor. An additional 5 vol of toluene was charged bringing the total volume to 10 vol. Temperature was adjusted to 20±5° C. 4-nitrobenzoyl chloride was charged to the reactor as a solid. Temperature was adjusted to 0±5° C. Triethylamine was charged slowly to maintain temperature at 0±5° C. Temperature was adjusted to 20±5° C. and reaction mixture was stirred for no less than 3 h. Upon reaction completion, 5 vol of a 2 M aqueous NaOH solution was added to the reactor, maintaining a temperature of 20±5° C., and the biphasic mixture was stirred for no less than 1 hour. Agitation was stopped, the phases were allowed to settle and the bottom, aqueous phase was drained off. A 5 vol solution of saturated ammonium chloride was added the reactor and the biphasic mixture was stirred for no less than 30 minutes. Agitation was stopped, the phases were allowed to settle and the bottom, aqueous phase was drained off. A 5 vol solution of saturated ammonium chloride was added the reactor and the biphasic mixture was stirred for no less than 30 minutes. 5 vol of water was charged to the reactor and the biphasic mixture was stirred for no less than 30 minutes. Phases were allowed to settle and the bottom, aqueous layer was drained off.

Organic phase was distilled to a total of 5 vol while maintaining an internal temperature at or below 45° C. 5 vol of toluene was charged and the mixture was distilled to 2.2 volumes. 1.2 vol of n-heptane was charged to the distilled toluene solution. Mixture was heated to an internal temperature of 70±5° C. and stirred for no less than 15 minutes and no more than 1 hour. Solution was cooled to 60±5° C. over 30 minutes. The clear solution was seeded with 0.010 w/w equiv of Compound VIII (4-nitrobenzoate ester) seeds and agitated for no less than 1 hour and no more than 2 h. 2.4 vol of n-heptane was charged at a linear rate over 5 hours. Slurry was cooled to 20±5° C. over 5 h. Slurry was aged for no less than 5 h. The solids were isolated via filtration. A 2 vol wash solution of 75:25 n-heptane:toluene was used to wash the wet cake. The wet cake was transferred to drying equipment and dried under vacuum at a temperature of 40±5° C. until a constant weight was observed. The proton NMR of the crude lactol was consistent with benzoate ester of compound VIII, 4-nitrobenzoate ester.

¹H-NMR CDCl₃: δ 8.21 (d, 2H, J=8 Hz); 8.10 (d, 2H, J=8 Hz); 6.93-6.88 (m, 1H); 6.81-6.76 (m, 1H) 6.73 (d, 1H, J=4 Hz); 4.09 (d, 1H, J=4 Hz); 3.95-3.91 (m, 1H); 3.91 (s, 3H); 1.60 (s, 3H); 0.86-0.84 (d, 3H, J=8 Hz) ppm.

### Step 5

### Synthesis of (2R,3S,4S,5R)-3-(3,4-difluoro-2-methoxyphenyl)-4,5-dimethyl-5-(trifluoromethyl)tetrahydrofuran-2-carbonitrile (VII)

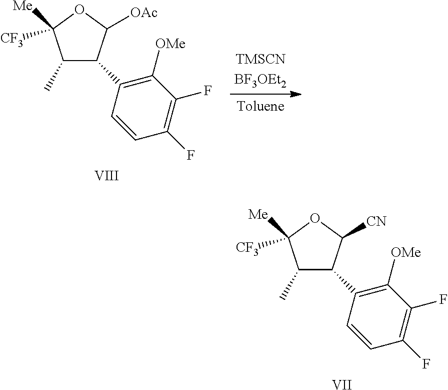 [SMILES: COc1c([C@H]2C(OC(C)=O)O[C@@](C)(C(F)(F)F)[C@H]2C)ccc(F)c1F ; "VIII" ; COc1c([C@H]2[C@H](C#N)O[C@@](C)(C(F)(F)F)[C@H]2C)ccc(F)c1F ; "VII"]

Transfered compound VIII (acetate ester) (1.87 L toluene solution containing 987 g of compound VIII obtained in the step above; 2.68 mols) to a 50 L jacketed reactor predried with N₂ gas/vacuum 4 times under N₂ gas. Anhydrous toluene (7.0 L) was added, stirred and cooled to −31° C. TMS-CN (385 g; 3.7 mols; 1.4 eq) was added over 8 minutes. BF₃ etherate (380 g=330 ml; 2.68 mols) was added dropwise over 6 minutes (Temp. −31.5° C. increased to −27.6° C.). The bath temperature was set at −21.6° C., and the reaction mixture was stirred for 2.5 hours. Potassium hydroxide (3.5 L of 2.0 M solution) was added over about 5 minutes (temperature rose to +8° C.). The bath temperature was raised to +20° C., the reaction mixture was stirred at +20° C. for about 10 minutes. The layers were separated and the aqueous layer was re-extracted with 6 L of toluene. The toluene solutions were re-extracted with 1.5 L of 2M potassium hydroxide before vacuum concentrating them to about 900 g of oil. The oil was diluted with 5 L of methanol and re-concentrated to give 887 g (2.65 mols, 98.7% is pure) of final crude compound VII. Proton NMR of the solid was consistent with the expected structure. NMR did not detect any methanol or toluene, suggesting that the solid is not solvated.

The crude compound VII was purified using a preparative silica gel column packed in 85:15 hexane:MTBE. A solution of 0.78 kg of the crude product in the packing solvent mixture was transferred to the column and eluted with the packing solvent mixture. The fractions were split approximately in half and each half was proceeded separately to the next compound (compound III). The early half of the fractions contained 333 g (or 993 mmols) of compound VII whereas the late half of the fractions contained 274 g (or 818 mmols) of compound VII. The example below shows the process of converting compound VII collected from the early half of the fractions. Compound VII obtained in the late half of the fractions was also converted to compound III following an identical process except for the amounts of reagents and solvents.

### Synthesis of (2R,3S,4S,5R)-3-(3,4-difluoro-2-methoxyphenyl)-4,5-dimethyl-5-(trifluoromethyl)tetrahydrofuran-2-carbonitrile (VII)

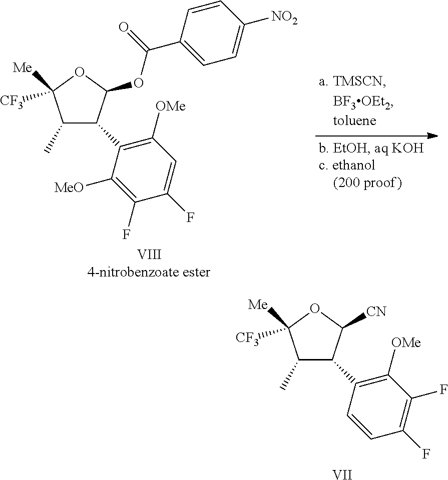

Compound VIII (4-nitrobenzoate ester) solids were charged to a reactor. 10 vol of toluene was charged to the reactor, and temperature was controlled to 20±5° C. 40% trimethylsilyl cyanide solution in toluene (1.2 equiv of TMSCN) was added to the reactor while maintaining a temperature of 20±5° C. Batch was cooled to −20±5° C. 1.0 equiv of boron trifluoride was added slowly while maintaining the temperature at −20±5° C. Batch was stirred at −20±5° C. for no less than 3 hours. Upon reaction completion batch was heated back to 20±5° C. 10 vol of 20% w/w aqueous potassium hydroxide solution was added to the batch, the biphasic mixture was stirred for no less than 1 h. 5 vol of ethanol was added to the batch and the biphasic mixture was stirred for no less than 12 h. Phases were allowed to settle, and the bottom aqueous layer was drained off. 10 vol of 20% w/w aqueous potassium hydroxide solution was added to the batch, the biphasic mixture was stirred for no less than 1 h. Phases were allowed to settle, and the bottom aqueous layer was drained off. 10 vol of 20% w/w aqueous potassium hydroxide solution was added to the batch, the biphasic mixture was stirred for no less than 30 minutes. Phases were allowed to settle, and the bottom aqueous layer was drained off. 10 vol of water was added to the batch and the biphasic mixture was stirred for no less than 30 minutes. Phases were allowed to settle, and the bottom aqueous layer was drained off. Top, organic phase was distilled to a total of 4 vol while maintaining internal temperature at or below 45° C. 7 vol of ethanol was charged and the mixture was distilled to a total of 4 vol. Another 7 vol of ethanol was charged and the mixture was distilled to a total of 4 vol. Distillation continued until residual toluene was no more than 1.0% w/w. The proton NMR of the crude lactol was consistent with benzoate ester of compound VII.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1H, J=7.7 Hz); 1.64 (d, 3H, J=1.3 Hz); 0.80 (dq, 3H, J=7.3, 2.3 Hz) ppm

### Step 6

### Synthesis of (2R,3S,4S,5R)-3-(3,4-difluoro-2-methoxyphenyl)-4,5-dimethyl-5-(trifluoromethyl)tetrahydrofuran-2-carboxylic acid (III)

### Procedure 1:

Compound VII (333.06 g, 993.4 mmol) was dissolved in 2.60 L methanol and 2.60 L of 2.0 M potassium hydroxide in a 25 L jacketed reactor under N₂ gas. The mixture was stirred at 55° C. for 20 hours. After overnight the slurry was a clear solution. Proton NMR indicated that the reaction complete. The reaction mixture was cooled to +15° C., and added 2925 ml of 2N HCl+5 L MTBE. After stirring for about 5 minutes, the layers were separated, and the aqueous was re-extracted with 5 L of MTBE. The MTBE solutions were combined and dried over magnesium sulfate, filtered and washed with MTBE. The combined MTBE solution was concentrated to an oil and re-concentrated with 4 L toluene, to give 360 g of an oily product.

The oil was dissolved in 4.0 L of anhydrous toluene, and stirred at 60° C. To the solution was added (R)-(+)-α-methylbenzylamine (142 ml, 1.1156 mol, 1.12 eq.) rapidly. The solution was seeded and after about 5 minutes the solution began to deposit crystals. The slurry was cooled to 45° C. and stirred for 1.5 hours before cooling it to 35° C. and stirring for additional 1.5 hours. The temperature was then lowered to 25° C. (for an additional 1.5 hours stirring) and finally to 15° C. and stirred overnight. The product was filtered and washed with 2×200 ml of toluene (at 15° C.) and dried overnight at 50° C., 1 mm, to give 431.37 g of (R)-(+)-α-methylbenzylamine salt of compound III.

### Procedure 2:

4.5 volumes of a 10% w/w solution of KOH was charged to a solution of Compound VII in 4vol ethanol at 20° C. The reaction mixture was heated to 55° C. and stirred or 12 h. Upon completion of the reaction, the mixture was cooled down to 20° C. and 5 volumes of toluene was charged. After 30 min of stirring, the phases were separated, and the organic phase was discarded. The aqueous phase was extracted with toluene and 9.5 volumes of 7% HCl solution. After 60 min of stirring the phases were separated and the aqueous phase was discarded. The organic phase was washed with 7.3 volumes of water, twcie. The organic phase was distilled down to 6 volumes under vacuum at an internal temperature below 40° C. 6 volumes of toluene were charged and the solution was distilled down to 5 volumes under vacuum at an internal temperature below 40° C., to afford compound III. The proton NMR of the crude lactol was consistent with compound III.

¹H-NMR d6-DMSO: δ 13.00 (s, 1H); 7.27-7.08 (m, 2H); 4.98 (d, 1H, J=10.5 Hz); 4.08 (dd, 1H, J=10.5, 7.6 Hz); 3.93 (d, 3H, J=2.1 Hz); 2.66 (p, 1H, J=7.5 Hz); 1.53 (d, 3H, J=1.4 Hz); 0.73-0.64 (m, 3H) ppm.

### Step 7

### Synthesis of quinine salt of (2R,3S,4S,5R)-3-(3,4-difluoro-2-methoxyphenyl)-4,5-dimethyl-5-(trifluoromethyl)tetrahydrofuran-2-carboxylic acid (IV)

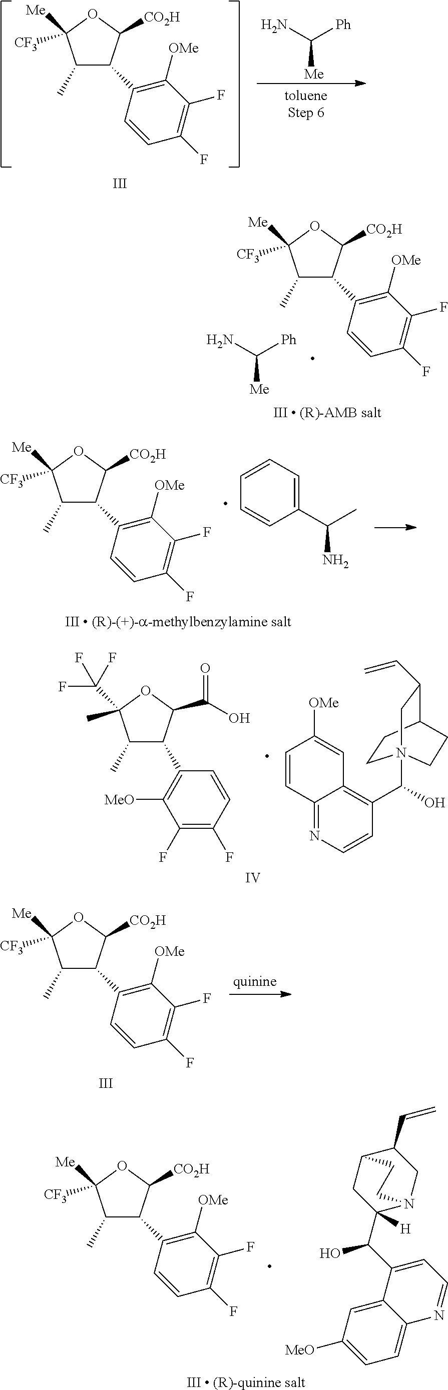

Compound III (2 g) was dissolved in 10 mL of toluene at 60° C. Separately, (R)-AMB (1.2 eq.) was dissolved in 2 mL of toluene and added to Compound III solution over 1 hour. The resulting solution was then cooled to 50° C. over 1 hour and held for 1 hour for the self-nucleation to happen. Then the slurry was cooled to 20° C. over 5 hours and agitated at 20° C. for about 8 hours and filtered under vacuum. The resulting wet cake was washed with a 5 mL of toluene and dried under vacuum at 40° C. to provide about 70% yield compared to input Compound III free form. The resulting solid was identified as (R)-(+)-α-methylbenzylamine salt of Compound III Form A from XRPD.

Form A of the (R)-(+)-α-methylbenzylamine salt of Compound III presents the following XRPD peaks in a standard X-Ray Powder Diffraction experiment.

TABLE 1

| No. | Pos. [°2θ] | Rel. Int. [%] | Priority |
|---|---|---|---|
| 1 | 10.32 | 28.04 | 2 |
| 2 | 11.78 | 11.26 | 3 |
| 3 | 12.81 | 15.58 | 3 |
| 4 | 13.44 | 57.18 | 1 |
| 5 | 14.01 | 5.72 | |
| 6 | 14.76 | 6.18 | |
| 7 | 15.91 | 18.58 | 3 |
| 8 | 17.00 | 5.45 | |
| 9 | 17.45 | 100 | 1 |
| 10 | 18.25 | 36.72 | 2 |
| 11 | 18.53 | 11.02 | |
| 12 | 18.95 | 63.98 | 1 |
| 13 | 21.96 | 11.86 | |
| 14 | 24.07 | 24.65 | 2 |
| 15 | 24.36 | 8.98 | |
| 16 | 24.62 | 7.83 | |
| 17 | 24.69 | 7.32 | |
| 18 | 25.09 | 8.2 | |
| 19 | 29.69 | 7.8 | |

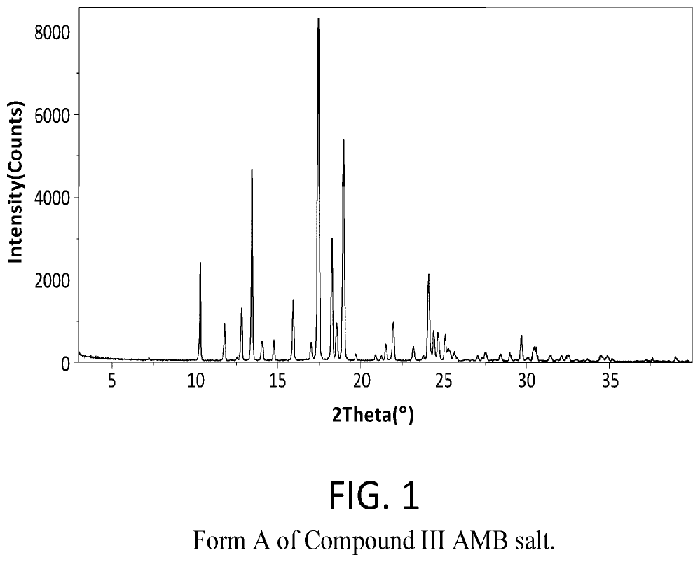

In Table 1, the term priority signifies perceive degree of importance of a particular diffraction peak based on its relative intensity. The skilled artisan would understand that the relative intensities of diffraction peaks can be affected by the experimental conditions. The overall diffraction pattern as shown in FIG. 1 may also be equally important for characterizing a solid form.

Isopropyl alcohol (27.8 L) was charged to a vessel and the solvent was saturated with N₂ gas. n-Heptane (47.2 L) was added to the vessel while continuously stirring the mixture and purging the vessel with N₂ gas. The n-heptane/isopropyl alcohol mixture was transferred to a clean drum. (R)-(+)-α-Methylbenzylamine salt of Compound III (10.42 Kg of) was added to the solvent mixture while purging with N₂ gas. The reactor was sealed and three vacuum/N₂ gas cycles were conducted. Methylene chloride (80 L) was added to the reaction mixture before slowly starting the agitator to suspend and slurry the solids. Hydrochloric acid (full carboy of 2.0 M) was added. Stirred the mixture for 15 minutes and let the phases separate (about 10 minutes). Compound III was in the lower phase. Separated the phases and then returned the organic phase to the reactor. Repeated the wash procedure with two more portions (19 L each) of 2 M HCl. Dried the lower organic phase by stirring with anhydrous magnesium sulfate (1 kg) and then filtered off the supernatant. Distilled the mixture down to a volume of about 11 L, and then added isopropanol (10 L) to the reactor. Repeated the distillation and isopropanol addition two more times. Added quinine (7.11 kg) to the mixture. Diluted the remaining solution with a mixture (57 L total) of isopropanol and n-heptane and then heated the resulting mixture to about 60 to 65° C. to dissolve all solids. Cooled to 45° C. over 90 min. Added quinine salt of compound IV seed material during the cooling period, when the mixture temperature was about 56-58° C. Cooled the mixture to 20° C. over 3 h. Stirred the mixture for 100 min and then filtered. Washed the filter cake with an isopropanol/n-heptane mixture (8 L) and then dried the solids at 75° C. The product was recrystallized from isopropanol/n-heptane to provide 8.7 kg of IV.

Quinine (1 eq.) was added to Compound III (2 g) in about 4 mL of dichloromethane. The resulting slurry/solution was then solvent swapped to 2-propanol (4 mL) and heated to 70° C. to obtain complete dissolution. The solution was then cooled to 65° C. and n-heptane (12 mL) was added over one hour. During this heptane addition, self-nucleation was observed, and the resulting slurry was stirred at 65° C. for 1 hour. The slurry was then cooled to 20° C. over 3 hours and stirred at 20° C. for 2 hours. The slurry was filtered and washed with 3 mL of 75 V % heptane in 2-propanol mixture. The wet solids were analyzed by XRPD and identified as the 2-propanol solvate of Compound III quinine salt Form A (i.e., Compound IV). The wet cake was then dried at 50° C. for 24 hours to obtain Compound IV Form A in about 85% yield.

Form A of Compound IV may be characterized by the following XRPD peaks in a standard X-Ray Powder Diffraction experiment.

TABLE 2

| No. | Pos. [°2θ} | Rel. Int. [%] | Priority |
|---|---|---|---|
| 1 | 5.91 | 12.52 | |
| 2 | 6.48 | 100 | 1 |
| 3 | 6.75 | 17.86 | |
| 4 | 7.16 | 83.17 | 1 |
| 5 | 7.48 | 36.2 | 3 |
| 6 | 8.17 | 15.77 | |
| 7 | 9.79 | 10.28 | |
| 8 | 9.93 | 13.88 | |
| 9 | 11.31 | 15.64 | |
| 10 | 11.65 | 10.77 | |
| 11 | 11.85 | 21.32 | |
| 12 | 12.61 | 11.76 | |
| 13 | 12.81 | 38.92 | 3 |
| 14 | 12.97 | 64.51 | 1 |
| 15 | 13.2 | 32.35 | |
| 16 | 13.48 | 16.95 | |
| 17 | 13.91 | 50.21 | 2 |
| 18 | 14.48 | 54.11 | 2 |
| 19 | 14.94 | 21.87 | |
| 20 | 15.25 | 29.27 | |
| 21 | 15.54 | 14.26 | |
| 22 | 15.66 | 10.61 | |
| 23 | 16.25 | 42.39 | 2 |
| 24 | 17.57 | 15.21 | |
| 25 | 18.30 | 10.74 | |
| 26 | 18.75 | 18.23 | |
| 27 | 19.12 | 42.79 | 3 |
| 28 | 19.79 | 16.98 | |
| 29 | 20.40 | 10.28 | |
| 30 | 20.75 | 15.48 | |
| 31 | 21.18 | 15.63 | |
| 32 | 24.57 | 12.24 | |
| 33 | 24.87 | 12.03 | |

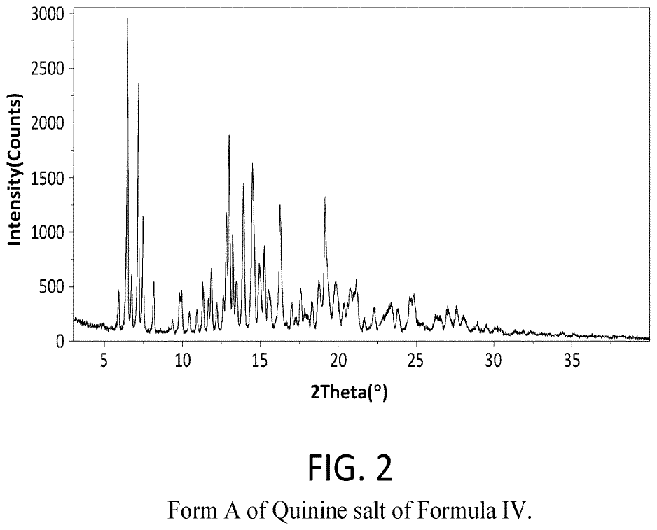

In Table 2, the term “priority” signifies perceived degree of importance of a particular diffraction peak based on its relative intensity. The skilled artisan would understand that the relative intensities of diffraction peaks can be affected by the experimental conditions. The overall diffraction pattern as shown in FIG. 2 may also be equally important for characterizing a solid form.

### Step 8

### Synthesis of methyl 4-((2R,3S,4S,5R)-3-(3,4-difluoro-2-methoxyphenyl)-4,5-dimethyl-5-(trifluoromethyl)tetrahydrofuran-2-carboxamido)picolinate (II)

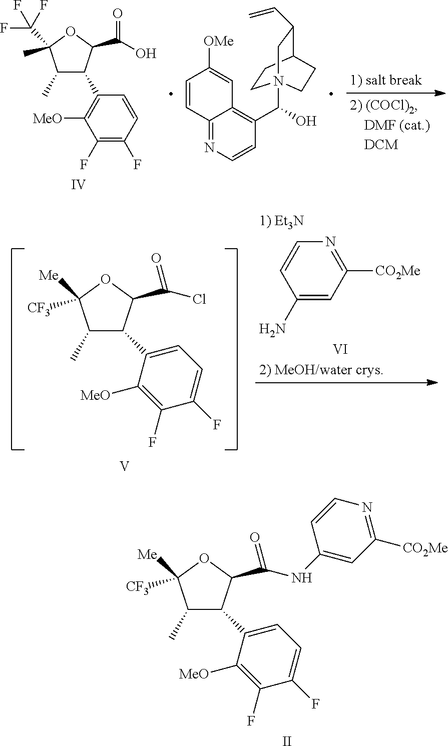

### Procedure 1:

Compound IV (quinine salt of compound III, 2.8 kg) was dried in a tray dryer ≤50° C. for at least 24 hours until the residual IPA content was ≤0.5% by ¹H NMR analysis. The dried quinine salt was charged to a 100 L jacketed reactor followed by addition of dichloromethane (DCM, 30.1 kg). A 2.5 M hydrochloric acid solution (9.0 kg) was charged and the mixture was agitated at 20±5° C. for at least 15 min. The layers were separated and the aqueous layer was discarded (this step was repeated two more times). The organic layer was washed with water (5.6 kg) and the layers were separated discarding the aqueous layer. A sample was pulled (IPC) from the organic layer in order to determine the amount of quinine. If the quinine was >1.0%, the organic layer was washed with aqueous hydrochloric acid and the layers were separated discarding the aqueous layer. The organic layer was transferred to a rotovap and distilled to 4 vol. DCM was charged to the 20 L rotovap round bottom flask (rbf) and the mixture was distilled to 4 vol. A sample was pulled (IPC) and analyzed for water content (KF) for information only.

The mixture was transferred to the 100 L jacketed reactor. N,N-Dimethylformamide (DMF, 33.1 g) was charged followed by oxalyl chloride (682.4 g) and the mixture was agitated at 20±5° C. for at least 3 h or until the amount of compound III present in the reaction mixture was <0.50%. The mixture was transferred to the 20 L rotovap rbf and distilled to 2 vol. DCM was charged to the rotovap rbf and the mixture was distilled to 3 vol (this step was repeated). A sample was pulled (IPC) and the amount of the oxalyl chloride was determined (derivative formation). When the amount of oxalyl chloride was determined to be >0.50%, DCM was charged to the mixture and the mixture was distilled to 3 vol (this step was repeated three times, if necessary, until the amount of oxalyl chloride was <0.50%).

Compound VI (690.7 g) was charged to the 100 L jacketed reactor followed by DCM. The mixture was agitated and the temperature of the mixture was adjusted to 10±5° C. Triethylamine (TEA, 501.5 g) was charged to the 100 L jacketed reactor followed by transfer of the contents of the 20 L rbf containing the acid chloride. (i.e., compound V). The temperature of the reactor was adjusted to 20° C. over at least 30 min and agitated at 20±5° C. for at least 3 h. A sample was pulled (IPC) in order to determine the amount of compound III. If compound III was present in an amount >1.5%, the reaction was allowed to continue for at least 1 h at 20±5° C. The process of pulling a sample and determining compound III content followed by stirring for at least 1 additional hour was repeated if necessary.

Water (11.2 kg) was charged to the reactor and the mixture was agitated for at least 15 min. The layers were allowed to separate and the aqueous layer was discarded. The organic layer was washed with an 18% aqueous citric acid solution (9.5 kg) followed by water (5.7 kg) and the aqueous layers discarded after each wash. The 100 L jacketed reactor was cleaned. The organic layer was transferred to the cleaned 100 L jacketed reactor and the mixture was distilled to 4 vol. Methanol was charged to the 100 L jacketed reactor and the mixture was distilled to 4 vol (this step was repeated). A sample was pulled (IPC) in order to determine the DCM content (¹H NMR) for information only. Additional Methanol (2.8 kg) was charged followed by water (4.9 kg). The temperature was adjusted to 60±5° C. and the mixture was agitated. The temperature of the mixture was adjusted to 55±5° C. and the mixture was agitated at 55±5° C. for at least 15 min. If needed Compound II seed was charged and the mixture was agitated at 55±5° C. for at least 30 min. Water (4.9 kg) was charged over the course of at least 5 h. The mixture was agitated at 55±5° C. for at least 30 min followed by adjustment of the temperature to 20±5° C. over at least 5 h. The mixture was agitated at 20±5° C. for at least 8 h. The solids were collected by filtration and washed with water/methanol. The product (1.7 kg) was dried and packaged.

### Procedure 2:

Compound IV (quinine salt of compound III; 10 g) was stirred with 60 mL toluene and 30 mL of aqueous hydrochloric acid solution (2 M) at 20° C. for over 30 minutes. The resulting emulsion was phase separated and organic phase (Compound III) was stirred with 30 mL of aqueous hydrochloric acid solution (2 M) at 20° C. for over 30 minutes. The resulting emulsion was phase separated and organic phase (with Compound III) was stirred with 20 mL of distilled water at 20° C. for over 30 minutes. The resulting emulsion was phase separated and organic phase (with compound III) was distilled down (by chasing with 60 mL toluene) to about 30 mL. To the latter solution, 30 mL of dichloromethane was charged along with 200 μL of N,N-dimethylformamide and resulting mixture stirred at 30° C. To this solution, a separately prepared oxalyl chloride (1.6 mL) mixed with 10 mL of dichloromethane, was added in slowly over 1 hour. Reaction was allowed to progress for over 3 hours to form Compound V. Post reaction, a series of put-take distillation cycles need to be performed (chasing with 100 mL toluene then 100 mL dichloromethane) to remove residual oxalyl chloride. Resulting solution is 50 mL of Compound V in dichloromethane, to which is charged (over 1 hour) a separately prepared solution of Compound VI (2.5 g) plus 40 mL dichloromethane plus 2.5 mL triethylamine. Reaction is allowed to happen at 25° C. for over 4 hours, after which a series of washes are performed [1) 40 mL water wash, 2) 26 mL citric acid solution wash, and 3) 20 mL water wash]. Solvent swap is performed via put-take distillation to exchange toluene with methanol, resulting in 70 mL of Compound II solution in methanol. A pre-mixed mixture of 12.5 mL methanol and 5 mL water is added to the latter solution. The batch is heated to 35 to 40° C. and seeded with Compound II crystals, following which a slow charge of 15 mL water over 3 hours and a cooldown to 20° C. for over 5 hours is carried out. The slurry is aged for over 8 hours and filtered under vacuum. The resulting wet cake was washed with a 30 vol % water in methanol solution and dried under vacuum at 40° C. to provide about 85 to 90% yield of Compound II. The isolated form is Form C.

### Step 9

### Synthesis of4-((2R,3S,4S,5R)-3-(3,4-difluoro-2-methoxyphenyl)-4,5-dimethyl-5-(trifluoromethyl)tetrahydrofuran-2-carboxamido)picolinamide (I)

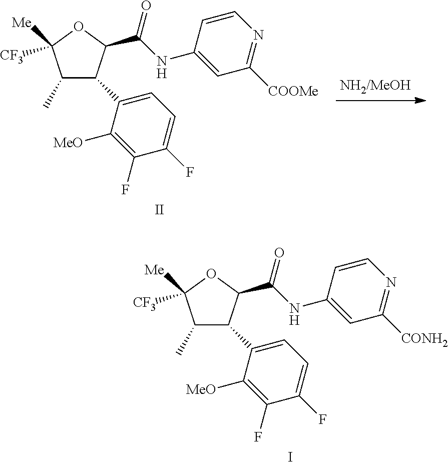

### Procedure 1:

Compound II (1.6 kg) was charged to a 100 L reactor followed by a 7 M solution of ammonia in methanol (10.7 kg). The temperature of the mixture was adjusted to 20±5° C. The mixture was agitated at 20±5° C. for at least 24 h or until the amount of the starting material present was <0.5% (an additional amount of ammonia solution may be added if necessary to reduce compound II to below 0.5%). The reaction mixture was transferred to a rotovap and distilled to 4 vol. Methanol was charged to the rotovap rbf and the mixture was distilled to 4 vol. The mixture was transferred to the 100 L reactor and methanol was charged. The temperature was adjusted to 55±5° C. and the mixture was agitated at 55±5° C. for at least 10 min. If a solution was not present in the reactor, the temperature of the mixture was adjusted to 60±5° C. and the mixture was agitated for at least 10 min followed by adjusting the temperature to 55±5° C. Water (7.9 kg) was charged over at least 2 hours to the rector while maintaining the temperature at 55±5° C. The mixture was agitated at 55±5° C. for at least 1 h followed by adjustment of the temperature to 20±5° C. over at least 12 h. The mixture was agitated at 20±5° C. for at least 5 hours and the solids were collected by filtration. The reactor was rinsed with methanol/water and the rinse was transferred through the filter cake. The solids were transferred to lined trays and dried in a tray drier at ≤45° C. for at least 12 h to yield 1.3 kg of crude compound (I).

### Procedure 2:

Ammonia gas is bubbled to a 30 vol % tetrahydrofuran in methanol solution to result in about 6 M concentrated solution. Compound II (10 g) is dissolved with 102 mLs of the prepared ammonia solution and reaction conducted at 20° C. for over 20 hours. The result is Compound I solution, to which 12.5 mL of water is charged slowly over 1 hour at 25° C. Seeding is carried with 0.5 wt % Compound I crystals at 25° C. and aged for 1 hour. 87.5 mLs of water is charged at 25° C. for over 4.5 hours. The slurry is aged for over 8 hours and filtered under vacuum. The resulting wet cake was washed with a methanol/tetrahydrofuran/water (35/15/50 volume ratio) and dried under vacuum at 40° C. to provide about 92 to 94% yield of Compound I. The isolated form is Form B.

### Step 10

### Purification of Compound I

### Procedure 1:

Crude compound I (1.23 kg) was charged to a 22 L rbf followed by acetone (5.01 kg). The mixture was agitated and the temperature was adjusted to 40±5° C. The mixture was polish filtered to a 20 L Jacketed Reactor. The temperature was adjusted to 35±5° C. and the mixture was agitated at 35±5° C. for at least 5 min. If the mixture was not a solution, the temperature was adjusted to 40±5° C. and the mixture was agitated at 40±5° C. for at least an additional 5 min. The temperature was adjusted to 35±5° C. and water (1.90 kg) was charged to the solution over the course of 1.5 h before seeding the solution. The mixture was agitated at 35±5° C. for at least 1 h. Water (2.49 kg) was charged to the reactor over at least 2 hours while maintaining a temperature of 35±5° C. and the mixture was agitated at 35±5° C. for at least 30 min. The temperature was adjusted to 20±5° C. over at least 5 h and the mixture was agitated at 20±5° C. for at least 5 h. The solid was collected by filtration and the reactor was rinsed with acetone/water sending the rinse through the filter cake. The solid was dried on the filter with N₂ gas for at least 30 min. The product was transferred to lined trays and dried in a tray drier to yield 1.10 kg of recrystallized compound (I).

### Procedure 2:

Compound I (10 g) obtained above was dissolved in 70 mL methanol and 30 mL tetrahydrofuran at 25° C. Polish filtration was then conducted. 12.5 mL of water was charged slowly over 1 hour at 25° C. Seeding was carried out with 0.5 wt % Compound I crystals at 25° C. and aged for 1 hour. 87.5 mLs of water was charged at 25° C. for over 4.5 hours. The slurry was aged for over 8 hours and filtered under vacuum. The resulting wet cake was washed with a methanol/tetrahydrofuran/water (35/15/50 volume ratio) and dried under vacuum at 40° C. to provide about 95% yield of Compound I. The isolated form was Form B.

### Example 2

### Synthesis of Compound III

Compound III may alternatively be synthesized in accordance with Scheme 2.

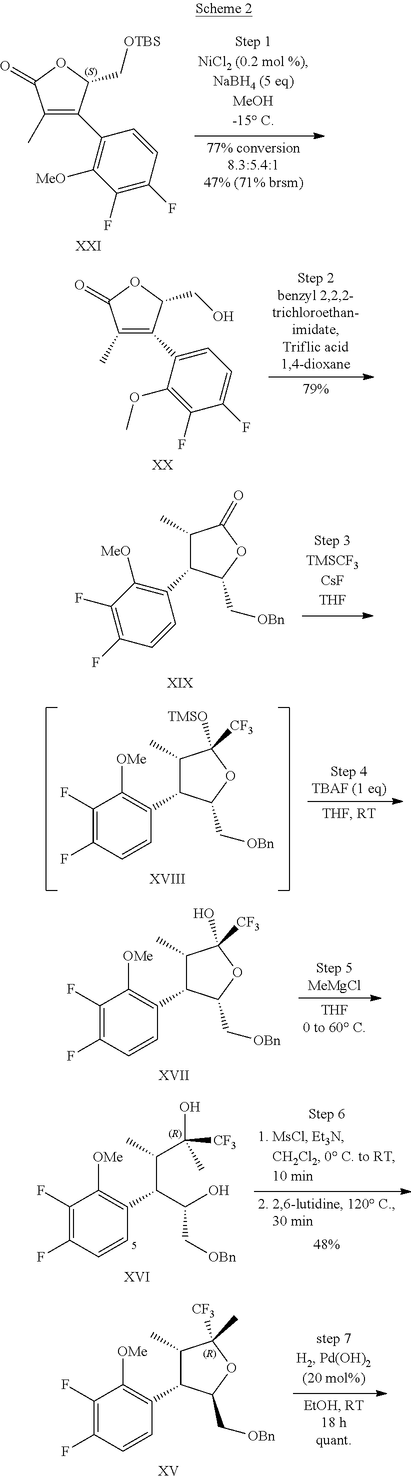

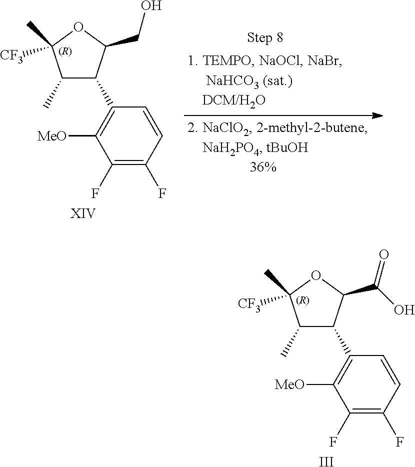

### Synthesis of (2R,3S,4S,5R)-3-(3,4-difluoro-2-methoxyphenyl)-4,5-dimethyl-5-(trifluoromethyl)tetrahydrofuran-2-carboxylic acid III

### Step 1

### Synthesis of (3S,4S,5S)-4-(3,4-difluoro-2-methoxy-phenyl)-5-(hydroxymethyl)-3-methyl-tetrahydrofuran-2-one (XX)

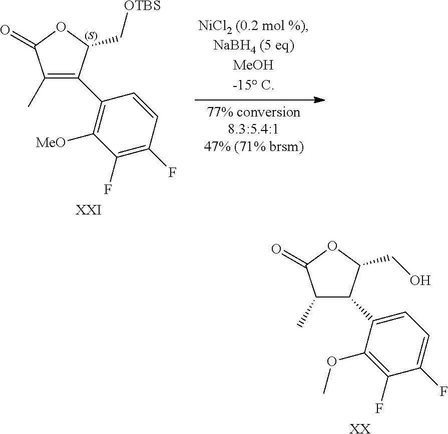

A solution of 5-(((tert-butyldimethylsilyl)oxy)methyl)-4-(3,4-difluoro-2-methoxyphenyl)-3-methylfuran-2(5H)-one XXI (1.064 g, 3.937 mmol) and NiCl₂·6H₂O (112.0 mg, 0.4712 mmol) in methanol (40 mL) was cooled to −15° C. (NaCl/ice). Then NaBH₄ (730 mg, 19.30 mmol) was added portionwise over 10 mins and on complete addition was stirred for a further 20 mins. The reaction mixture was poured into cool NH₄Cl (sat) solution, the phases were separated and aqueous phase further extracted with DCM (×2). The combined organic phases were washed with water and brine. The crude product was purified by silica gel chromatography (eluting from 0% to 30% EtOAc in heptane) to afford (3S,4S,5S)-4-(3,4-difluoro-2-methoxy-phenyl)-5-(hydroxymethyl)-3-methyl-tetrahydrofuran-2-one XX (501 mg, 47%). ¹H NMR (400 MHz, Chloroform-d) δ 6.81-6.69 (m, 1H), 6.64 (ddd, J=8.7, 5.6, 2.2 Hz, 1H), 4.74-4.62 (m, 1H), 3.91 (d, J=3.4 Hz, 3H), 3.52 (dd, J=12.1, 7.9 Hz, 1H), 3.28 (dd, J=12.1, 4.6 Hz, 1H), 3.14-2.92 (m, 1H), 1.84 (d, J=1.8 Hz, 1H), 0.93 (d, J=7.2 Hz, 3H) ppm. ESI-MS m/z calc. 272.08603, found 273.4 (M+1)+; Retention time: 1.32 minutes.

### Step 2

### Synthesis of (3S,4S,5S)-5-(benzyloxymethyl)-4-(3,4-difluoro-2-methoxy-phenyl)-3-methyl-tetrahydrofuran-2-one (XIX)

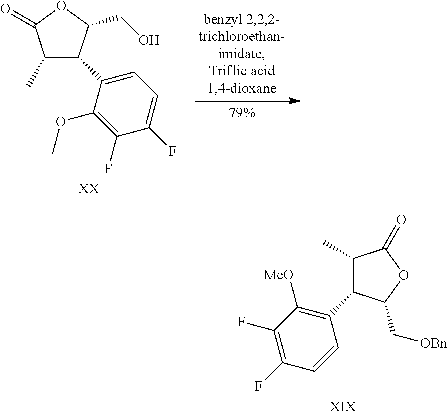

To a solution of (3S,4S,5S)-4-(3,4-difluoro-2-methoxy-phenyl)-5-(hydroxymethyl)-3-methyl-tetrahydrofuran-2-one XX (17.7 g, 65.02 mmol) and benzyl 2,2,2-trichloroethanimidate (34 g, 134.6 mmol) in 1,4-dioxane (200 mL), trifluoromethanesulfonic acid (1 mL, 11.30 mmol) was added dropwise at room temperature (a mild exotherm develops even with slow addition, it reaches 28° C.) and the reaction cooled with an ice bath to keep the addition going. The mixture was stirred overnight at room temperature. The solution was diluted with TBME and quenched by addition of NaOH 1M. The phases were separated and the organic phase was washed twice more with NaOH 2M. The combined organic extracts were washed with water, brine, dried over MgSO₄, filtered and concentrated in vacuo affording crude product. The crude product was purified by silica gel chromatography (220 g column. Gradient: 0 to 20% EtOAc in heptane) to afford impure product. Product was dissolved in DCM and washed twice with NaOH 2 M, then water, dried (MgSO₄), filtered and concentrated in vacuo to provide (3S,4S,5S)-5-(benzyloxymethyl)-4-(3,4-difluoro-2-methoxy-phenyl)-3-methyl-tetrahydrofuran-2-one XIX (18.7 g, 79%). ¹H NMR (400 MHz, Chloroform-d) δ 7.34-7.27 (m, 1H), 7.31-7.17 (m, 2H), 7.21-7.13 (m, 2H), 6.79 (td, J=9.1, 7.2 Hz, 1H), 6.70 (ddd, J=8.5, 5.7, 2.1 Hz, 1H), 4.87-4.78 (m, 1H), 4.44 (d, J=11.8 Hz, 1H), 4.28 (d, J=11.9 Hz, 1H), 3.89 (d, J=3.2 Hz, 3H), 3.51 (dd, J=10.3, 6.8 Hz, 1H), 3.25 (s, 1H), 3.04 (s, 1H), 0.97 (d, J=7.1 Hz, 3H) ppm; ESI-MS m/z calc. 362.13297, found 363.4 (M+1)+; Retention time: 0.95 minutes.

### Steps 3 and 4

### Synthesis of (2S,3S,4S,5S)-5-(benzyloxymethyl)-4-(3,4-difluoro-2-methoxy-phenyl)-3-methyl-2-(trifluoromethyl)tetrahydrofuran-2-ol (XVII)

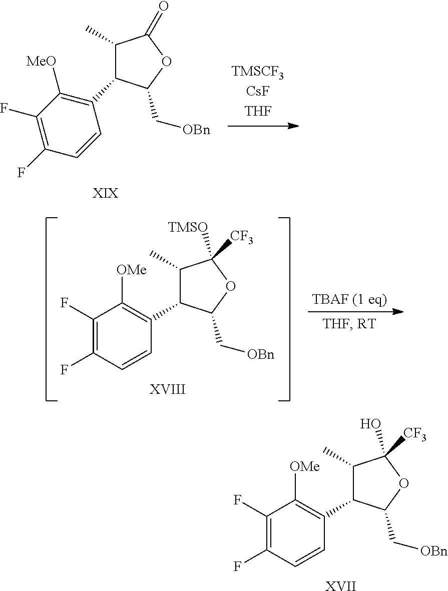

(3S,4S,5S)-5-(benzyloxymethyl)-4-(3,4-difluoro-2-methoxy-phenyl)-3-methyl-tetrahydrofuran-2-one XIX (948 mg, 2.616 mmol) was loaded into a round-bottomed flask and backfilled with N₂ gas/vacuum 3 times. Trimethyl(trifluoromethyl)silane (1.7 mL, 11.50 mmol) was added using a syringe. THF (0.4 mL) was added followed by anhydrous CsF (97.5 mg, 0.6419 mmol) (oven dried). The mixture was backfilled with N₂ gas/vacuum three times. The resulting mixture was stirred at room temperature over the weekend. Additional THF (9.5 mL) was added followed by TBAF (2.6 mL of 1 M, 2.600 mmol) at room temperature and stirred for 10 minutes. The crude mixture was dissolved in DCM and wash with water (twice) and brine. The solvent was removed in vacuo to leave crude (2S,3S,4S,5S)-5-(benzyloxymethyl)-4-(3,4-difluoro-2-methoxy-phenyl)-3-methyl-2-(trifluoromethyl)tetrahydrofuran-2-ol XVII (367 mg, 32%). ¹H NMR (500 MHz, Chloroform-d) δ 7.25-7.12 (m, 3H), 7.12-7.02 (m, 2H), 6.72 (dtt, J=11.1, 7.0, 3.8 Hz, 2H), 4.47 (qd, J=6.1, 2.3 Hz, 1H), 4.35 (d, J=11.9 Hz, 1H), 4.22-4.14 (m, 1H), 3.98 (dd, J=8.5, 6.0 Hz, 1H), 3.76 (t, J=1.6 Hz, 3H), 3.46-3.35 (m, 1H), 3.20 (ddd, J=10.1, 5.8, 2.1 Hz, 1H), 2.95 (p, J=7.5 Hz, 1H), 1.27-1.11 (m, 3H), 0.81 (td, J=7.0, 2.1 Hz, 1H), 0.75 (dd, J=7.2, 1.7 Hz, 3H) ppm; ESI-MS m/z calc. 432.136, found 431.5 (M−1)−; Retention time: 1.01 minutes.

### Step 5

### Synthesis of (2S,3S,4S,5S)-6-benzyloxy-4-(3,4-difluoro-2-methoxy-phenyl)-1,1,1-trifluoro-2,3-dimethyl-hexane-2,5-diol (XVI)

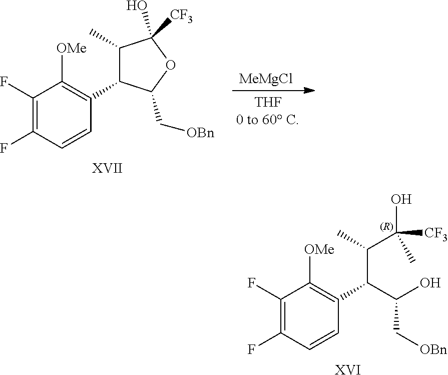

(2S,3S,4S,5S)-5-(benzyloxymethyl)-4-(3,4-difluoro-2-methoxy-phenyl)-3-methyl-2-(trifluoromethyl)tetrahydrofuran-2-ol XVII (415 mg, 0.9598 mmol) was dissolved in THF (8.5 mL) and cooled to 0° C. before adding MeMgCl (1.6 mL of 3 M, 4.800 mmol) dropwise. The reaction mixture was allowed to reach room temperature and then heated in the sealed vial to 60° C. until the reaction was complete. The reaction was allowed to cool to room temperature and quenched by addition of HCl 2M, extracted with EtOAc, dried over MgSO₄, filtered and concentrated in vacuo. The crude product was purified by silica gel chromatography (12 g column. Gradient: 0 to 20% EtOAc in heptane) to afford desired product, (2R,3S,4S,5S)-6-benzyloxy-4-(3,4-difluoro-2-methoxy-phenyl)-1,1,1-trifluoro-2,3-dimethyl-hexane-2,5-diol XVI (300 mg, 70%). ¹H NMR (400 MHz, Chloroform-d) δ 7.30-7.12 (m, 7H), 6.74 (td, J=9.3, 7.5 Hz, 1H), 4.33 (d, J=3.2 Hz, 2H), 4.30-4.21 (m, 1H), 3.88 (d, J=2.7 Hz, 3H), 3.47 (dd, J=7.3, 2.8 Hz, 1H), 3.29 (dd, J=9.3, 3.7 Hz, 1H), 2.91 (t, J=8.9 Hz, 1H), 2.42 (s, 1H), 2.37-2.25 (m, 1H), 2.19 (q, J=6.1, 5.1 Hz, 1H), 1.28 (d, J=1.2 Hz, 3H), 1.24 (dq, J=7.3, 1.8 Hz, 3H) ppm; EST-MS m/z calc. 448.1673, found 447.5 (M+1)+; Retention time: 0.96 minutes.

### Step 6

### Synthesis of (2R,3S,4S,5R)-5-(benzyloxymethyl)-4-(3,4-difluoro-2-methoxy-phenyl)-2,3-dimethyl-2-(trifluoromethyl)tetrahydrofuran (XV)

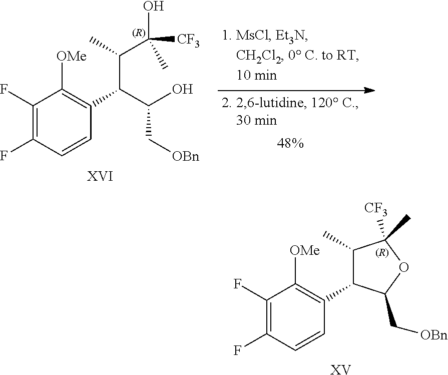

(2R,3S,4S,5S)-6-benzyloxy-4-(3,4-difluoro-2-methoxy-phenyl)-1,1,1-trifluoro-2,3-dimethyl-hexane-2,5-diol XVI (218 mg, 0.4861 mmol) and Et₃N (0.34 mL, 2.439 mmol) were dissolved in DCM (2.5 mL) and cooled to 0° C. using an ice/water bath. Then mesyl chloride (0.12 mL, 1.521 mmol) was added dropwise and the reaction stirred at 0° C. until reaction deemed complete. The reaction was quenched by the addition of MeOH (1.5 mL). The cold mixture was allowed to warm to room temperature and water (20 mL) was added. The layers were separated and the aqueous layer was extracted with DCM (2×15 mL). The combined organic phases were dried over Na₂SO₄, filtered and concentrated under reduced pressure to give the crude mesylate intermediate as yellow oil, 264 mg. The crude mesylated product was diluted in 2,6-lutidine (5 mL) and heated at 120° C. for 1 hour. The mixture was diluted with water (20 mL) and DCM (20 mL). The layers were separated and the aqueous layer was extracted with DCM (3×20 mL). The combined organic phases were washed with saturated CuSO₄ aq. (20 mL×4), dried over Na₂SO₄, filtered and concentrated under reduced pressure and the crude product purified by silica gel chromatography using a gradient of 0 to 10% EtOAc in heptane then 20% EtOAc:80% heptane to provide (2R,3S,4S,5R)-5-(benzyloxymethyl)-4-(3,4-difluoro-2-methoxy-phenyl)-2,3-dimethyl-2-(trifluoromethyl)tetrahydrofuran XV (100 mg, 48%). ¹H NMR (500 MHz, Chloroform-d) δ 7.28-7.11 (m, 5H), 6.76-6.56 (m, 2H), 4.61-4.38 (m, 3H), 3.90-3.75 (m, 4H), 3.57 (ddd, J=11.0, 2.6, 1.4 Hz, 1H), 3.42 (ddd, J=11.1, 4.7, 1.4 Hz, 1H), 2.52 (pd, J=7.7, 1.4 Hz, 1H), 1.48-1.41 (m, 3H), 0.67 (dt, J=7.2, 2.2 Hz, 3H) ppm. ESI-MS m/z calc. 430.15674, found 431.5 (M+1)+; Retention time: 1.21 minutes.

### Step 7

### Synthesis of [(2R,3S,4S,5R)-3-(3,4-difluoro-2-methoxy-phenyl)-4,5-dimethyl-5-(trifluoromethyl)tetrahydrofuran-2-yl]methanol (XIV)

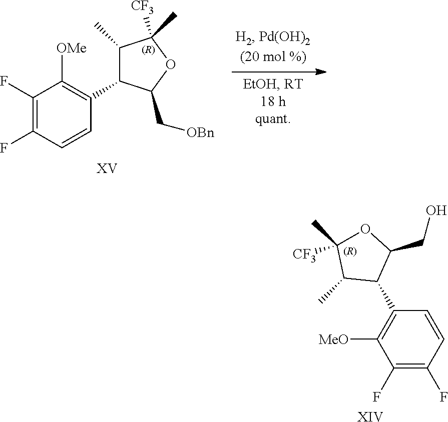

Pd(OH)₂ (Degussa, 23.7 mg, 0.03375 mmol) was loaded into a N₂ gas filled round bottomed flask. The flask was backfilled with N₂ gas three times. Then (2R,3S,4S,5R)-5-(benzyloxymethyl)-4-(3,4-difluoro-2-methoxy-phenyl)-2,3-dimethyl-2-(trifluoromethyl)tetrahydrofuran XV (76 mg, 0.1766 mmol) in EtOH (2 mL) was added and again the flask was backfilled with N₂ gas (×3). A balloon of Hydrogen was bubbled through the resulting solution until empty. The balloon was refilled and the mixture vigorously stirred overnight at room temperature. The reaction flask was flushed with N₂ gas three times (vacuum/N₂ gas cycles). The catalyst was then filtered-off by passing the mixture through pre-wetted celite cartridge with EtOH and solvent removed in vacuo to provide [(2R,3S,4S,5R)-3-(3,4-difluoro-2-methoxy-phenyl)-4,5-dimethyl-5-(trifluoromethyl)tetrahydrofuran-2-yl]methanol XIV (68 mg, quantitative yield based on ˜94% purity). ¹H NMR (400 MHz, Chloroform-d) δ 6.76 (td, J=9.2, 7.3 Hz, 1H), 6.67 (ddd, J=8.6, 5.6, 2.1 Hz, 1H), 4.46 (ddd, J=11.3, 4.0, 2.5 Hz, 1H), 3.92 (d, J=2.5 Hz, 3H), 3.89-3.70 (m, 2H), 3.41 (dd, J=12.3, 4.0 Hz, 1H), 2.56 (p, J=7.7 Hz, 1H), 2.12-1.83 (m, 1H), 1.46 (d, J=1.4 Hz, 3H), 0.69 (dq, J=7.4, 2.4 Hz, 3H) ppm. EST-MS m/z calc. 340.10977, found 358.5 (M+1)+; Retention time: 0.94 minutes.

### Step 8

### Synthesis of (2R,3S,4S,5R)-3-(3,4-difluoro-2-methoxyphenyl)-4,5-dimethyl-5-(trifluoromethyl)tetrahydrofuran-2-carboxylic acid (III)

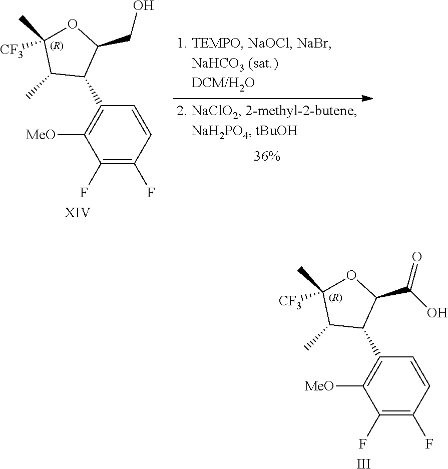

[(2R,3S,4S,5R)-3-(3,4-difluoro-2-methoxy-phenyl)-4,5-dimethyl-5-(trifluoromethyl)tetrahydrofuran-2-yl]methanol XIV (45 mg, 0.1322 mmol) was dissolved in CH₂Cl₂ (0.5 mL) and cooled to 0° C. NaBr (8.6 mg, 0.08358 mmol), tetrabutylammonium bromide (45.3 mg, 0.1405 mmol), TEMPO (7.7 mg, 0.04928 mmol) and NaHCO₃ aqueous sat. (0.4 mL) were then added. The resulting mixture was treated with NaOCl (9 μL, 0.1326 mmol) stirring vigorously and allowing to warm up to room temperature over 1 hour. HCl (0.13 mL of 1 M, 0.1300 mmol) was added until neutralizing pH to 6-7. Then tBuOH (2 mL), 2-methylbut-2-ene (0.5 mL of 2 M, 1.000 mmol) were added followed by an aqueous solution of NaClO₂ (12.6 mg, 0.1393 mmol) and NaH₂PO₄ (0.02 mL, 0.3192 mmol) and the mixture stirred at room temperature for 1-2 hours. The mixture was diluted with saturated aqueous NaH₂PO₄ solution (5 mL), and extracted with EtOAc (3×10 mL). The organic layers were combined and dried over MgSO₄, filtered and concentrated in vacuo to leave crude product. Product was purified by reverse-phase HPLC. Method: C18 Waters X-bridge column (19×150 mm, 5 micron), Gradient: MeCN in H₂O with 0.1% Ammonium hydroxide. 19 mL/min plus 1 mL/min MeCN at column dilution injection, Gradient 15.8% to 30.5% over 9 mins to afford (2R,3S,4S,5R)-3-(3,4-difluoro-2-methoxy-phenyl)-4,5-dimethyl-5-(trifluoromethyl)tetrahydrofuran-2-carboxylic acid III (17 mg, 36%). ¹H NMR (400 MHz, Methanol-d4) δ 7.11 (ddd, J=8.3, 5.6, 2.2 Hz, 1H), 6.96 (ddd, J=9.9, 8.9, 7.5 Hz, 1H), 4.96 (d, J=10.5 Hz, 1H), 4.16 (dd, J=10.5, 7.8 Hz, 1H), 3.99 (d, J=2.4 Hz, 3H), 2.73 (p, J=7.6 Hz, 1H), 1.59 (d, J=1.2 Hz, 3H), 0.86-0.68 (m, 3H) ppm. ESI-MS m/z calc. 354.08905, found 353.4 (M−1)−; Retention time: 0.59 minutes.

### Example 3

### Synthesis of Compound X

Compound X may alternatively be synthesized in accordance with Scheme 4.

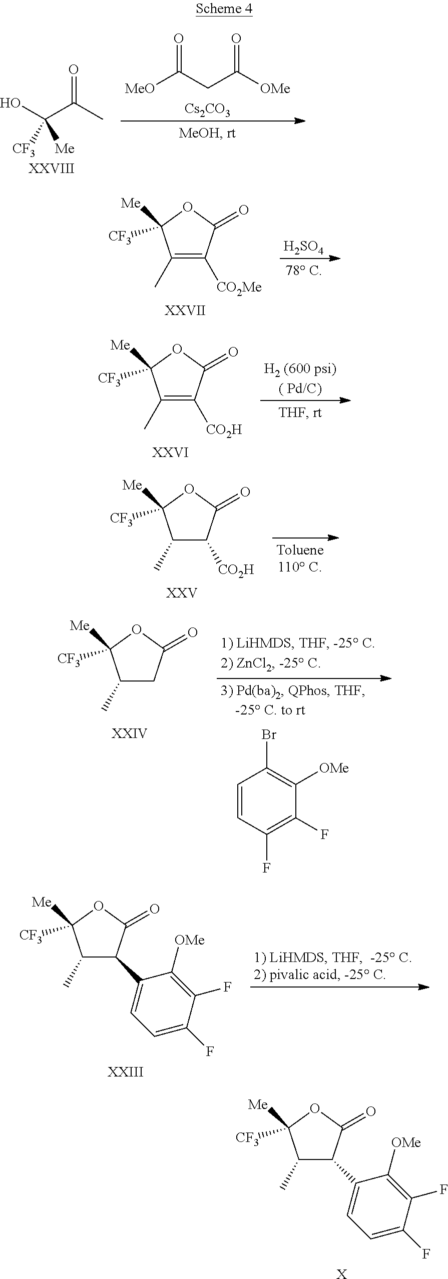

In some embodiments, Compound XXVI may be decarboxylated followed by hydrogenation reaction to afford Compound XXIV.

### Step 1

### Synthesis of methyl (R)-4,5-dimethyl-2-oxo-5-(trifluoromethyl)-2,5-dihydrofuran-3-carboxylate (XXVII)

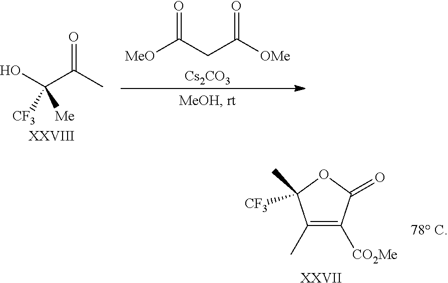

### Procedure 1:

A 10 L vessel, equipped with overhead-stirrer, nitrogen-inlet and temperature probe, was flushed with nitrogen and charged with 200.14 g (1.282 mol) of Compound XXVIII, 450 mL (520 g, 3.94 mol, 3.07 equiv.) of dimethyl malonate and 6.0 L of methanol. To the clear solution, 1003.16 g (3.079 mol, 2.4 equiv.) of cesium carbonate was added in portions. The addition was slightly exothermic and the internal temperature rose from 22° C. to 29° C. (when all Cs2CO3 had been added). A white thin suspension was obtained. In the course of 1.5 h the mixture became a clear colorless solution. The mixture was stirred over the weekend under nitrogen at RT.

An orange-brown solution was obtained. The solution was cooled to 2-3° C. and 3N HCl(aq) was added until pH 6-7. About 1300 mL of 3N HCl(aq) was required. Most of the methanol was removed under reduced pressure (water-bath at 52° C., down to 170 mbar). The left-over liquid was extracted with EtOAc (1.5 L, 3× 500 mL). The combined organic phase was washed with brine (500 mL) and dried over Na₂SO₄. After filtration, the solvent was removed under reduced pressure.

A brown oil was obtained, yield: 362 g. NMR 1 (CDCl₃): 18% w/w dimethyl malonate present. The material was purified by fractional distillation at 9-12 mbar to afford 256 g (1.075 mol, 84%) of Compound XXVII.

### Procedure 2:

To a solution of Compound XXVIII (249.5 g, 1.2786 mol), dimethyl malonate (509.9 g, 3.8595 mol) in methanol (6 L), cesium carbonate (1000 g, 3.0692 mol) was added portion-wise over 1 h. The mixture was stirred overnight. The mixture was cooled to 5° C. before adding 1% aqueous hydrochloric acid portion-wise, so that the temperature did not exceed 10° C. until pH 6-7 was obtained. The methanol was removed in vacuo and the resultant solution extracted with ethyl acetate (2×1.5 L). The combined organic extracts were washed with brine (1 L), dried (sodium sulphate) and concentrated. The reaction was repeated 3 times on the above scale, once on 0.664 of this scale and all batches combined to give Compound XXVII (283.2 g, 93%) as a black oil, which was used in the following step without purification. The oil was contaminated with 20% dimethyl malonate and 3% toluene, based on ¹H NMR.

¹H NMR (400 MHz, CDCl3) δ 3.90 (s, 3H), 2.44 (s, 3H), 1.70 (s, 3H) ppm.

### Step 2

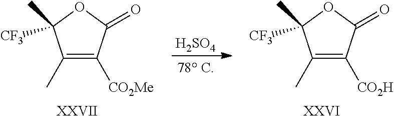

### Synthesis of (R)-4,5-dimethyl-2-oxo-5-(trifluoromethyl)-2,5-dihydrofuran-3-carboxylic acid (XXVI)

### Procedure 1:

A 9 L jacketed glass reactor was loaded with 5.7 L (10.43 kg, 106.4 mol, 44.5 equiv.) of sulfuric acid >95% and 569.0 g (2.389 mol) of Compound XXVII. The colorless solution was heated to 78-79° C. After 4 h, a dark-orange clear solution was obtained. A sample was mixed with ice and DCM, the separated organic phase washed with water, dried over Na₂SO₄ and concentrated for an NMR aliquote (¹H, ¹⁹F CDCl₃): 34-35% conversion. The mixture was stirred overnight at 78-79° C. After 21 h the internal temperature was 78.7° C., the mixture had become a little darker. A sample was mixed with ice and DCM, the separated organic phase washed with water, dried over Na₂SO₄ and concentrated for an NMR aliquote (¹H, ¹⁹F CDCl₃): 94% conversion. The mixture was cooled to 0-3° C. (took 2 h). The mixture was siphoned into a stirred mixture of 20 kg of ice and 9 L of DCM (transfer took 4-5 min.). The mixture was stirred vigorously for 10 min and the phases were allowed to settle. After phase separation, the aqueous phase was extracted with DCM (3×3 L, 3×2 L). The combined organic phase was washed with water (3.0 L), brine (1.5 L) and dried over Na₂SO₄. The organic phase was filtered and concentrated at reduced pressure (water bath at 55° C., down to 42 mbar) to give a light-beige solid, 525 g. The crude product was dissolved in TBME (2.2 L). To the solution, 3.8 L of sat. aq. NaHCO₃ was added carefully (evolution of gas and foaming). A thick suspension was obtained, addition of 3.5 L of water gave a clear two-phase system. The phases were separated and the organic phase was extracted with ½ sat. aq. NaHCO₃ (650 mL). The combined aqueous phase was washed with TBME (2×400 mL) and acidified with conc. aq. HCl to pH 1-2. A pinkish suspension was obtained which would become more opaque. The opaque mixture was extracted with TBME (7×1 L). The combined organic phase was washed with brine (200 mL) and dried over Na₂SO₄. After filtration, the solvent was removed at reduced pressure (water bath at 61° C., down to 8 mbar) to give a cream-colored solid of Compound XXVII. Yield 489.5 g (2.184 mol, 91.4%). NMR (¹H, ¹⁹F CDCl₃): 97.2% pure by ¹⁹F NMR, no TBME left. HPLC-MS (ACN): 98.12% pure, some decarboxylated product is present.

### Procedure 2:

A solution of Compound XXVII (284.8 g, 920.79 mmol) in concentrated sulphuric acid (2.5760 kg, 1.4 L, 26.264 mol) was stirred at 78° C. overnight. The reaction was cooled to 0° C. and poured onto ice (6 kg) with vigorous stirring. The mixture was extracted with dichloromethane (3×2 L). The combined organic extracts were washed with brine (1 L) and concentrated. The residue was taken up in toluene (2 L) and partially concentrated to a volume of 0.5 L. The solution was cooled to 0° C. and filtered. The product was washed with cold toluene (0.2 L) followed by heptane (0.3 L) and air dried. The reaction was repeated 5 times and all material combined to give Compound XXVI (786.4 g, 62%) as an off-white solid.

¹H-NMR (400 MHz, CHLOROFORM-D) δ 2.60 (d, 3H), 1.77 (d, 3H) ppm, acid proton not observed.

The combined mother liquors were concentrated to give a dark oil, which was treated with concentrated sulphuric acid (1.8400 kg, 1 L, 18.760 mol) and stirred at 78° C. overnight. The reaction was cooled to 0° C. and poured onto ice (4 kg) with vigorous stirring. The mixture was extracted with dichloromethane (3×1.5 L). The combined organic extracts were washed with brine (1 L) and concentrated. The residue was taken up in toluene (2 L) and partially concentrated to a volume of 0.5 L. The solution was cooled to 0° C. and filtered. The product was washed with cold toluene (0.2 L) followed by heptane (0.3 L) and air dried to give Compound XXVI (226.5 g, 18%) as a light brown solid.

¹H-NMR (400 MHz, CHLOROFORM-D) δ 2.61 (d, 3H), 1.77 (d, 3H) ppm, acid proton not observed.

### Steps 3 and 4

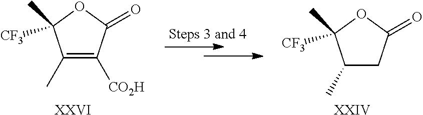

### Step 3

### Synthesis of (R)-4,5-dimethyl-5-(trifluoromethyl)furan-2(5H)-one (XXIX)

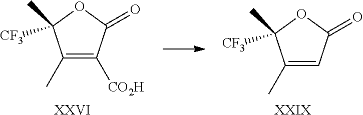

A 250 mL 3-neck RBF, equipped with a temperature probe and an air-cooled tube connected to a bubble-counter, was charged with 76.29 g (340.4 mmol) of Compound XXVI. The solid was melted (heating mantle was set at 120° C.). Once all solid had melted (melting at 95-97° C.) the heating was set at 175° C. and the internal temperature reached 168-199° C. and a steady evolution of gas was observed. After 30 min, the heating was set at 170° C. to maintain an internal temperature of 165-166° C. From that time, the evolution of gas slowly increased and at 45 min after the evolution of gas had started, the evolution of gas increased quite a bit and the mixture turned red-brown in 10-15 seconds. After the color change, the evolution of gas ceased abruptly. The liquid was cooled to RT. Isolated yield 60.62 g (336.5 mmol, 98.9%) of (R)-4,5-dimethyl-5-(trifluoromethyl)furan-2(5H)-one (XXIX).

### Step 4

### Hydrogenation of (R)-4,5-dimethyl-5-(trifluoromethyl)furan-2(5H)-one

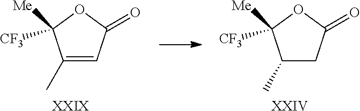

Pd/C (8.16 g, 7.668 mmol) was loaded into a RBF under N₂. EtOH (140 mL) was added followed by Compound XXIX (13.9479 g, 77.43 mmol). The flask was purged by 3 cycles of N₂/vacuum. A balloon filled with H₂ was connected with a 3-way valve adapter and the suspension was backfilled with H₂/vacuum 3 times. Then it was stirred under H₂ balloon until completion by TLC. The H₂ filled balloon was removed and the reaction was placed under vacuum to remove the hydrogen. 3 cycles of N₂/vacuum were performed and the suspension was then filtered through a Celite pad under a stream of N₂ using an inverted funnel. The solvent was removed leaving an orange oil confirmed to be one single diastereomer by NMR, affording 12.17 g of Compound XXIV, containing 1.1% of residual EtOH.

¹H NMR (400 MHz, Chloroform-d) δ 2.73-2.64 (m, 1H), 2.64-2.54 (m, 1H), 2.54-2.42 (m, 1H), 1.62 (q, J=1.0 Hz, 3H), 1.29 (dt, J=6.7, 2.2 Hz, 3H) ppm.

### Alternate Step 3

### Synthesis of (3S,4S,5R)-4,5-dimethyl-2-oxo-5-(trifluoromethyl)tetrahydrofuran-3-carboxylic acid (XXV)

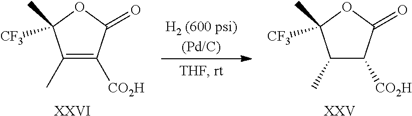

A slurry of palladium on carbon (101 g, 5% w/w, 47.453 mmol) in toluene (100 mL) was added to a solution of Compound XXVI (1012 g, 4.4474 mol) in THF (5 L) in an autoclave. The mixture was charged with hydrogen to 600 psi and stirred at room temperature for 18 h. The reaction was filtered and the filtrate concentrated to afford crude Compound XXV as a colorless oil which was contaminated with THF (5.11% w/w), the methyl ester of the desired product (1.92% w/w) and the isopropyl ester of the desired product (15.6% w/w) based on ¹H NMR.

### Alternate Step 4

### Synthesis of (4S,5R)-4,5-dimethyl-5-(trifluoromethyl)dihydrofuran-2(3H)-one (XXIV)

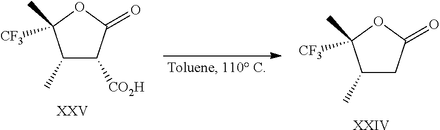

A solution of Compound XXV (1.124 kg, 3.8454 mol) in toluene (6 L) was heated at 110° C. for 42 h. On cooling the solution was concentrated. Vacuum distillation (92-96° C., 18 millibar) gave Compound XXIV (625 g, 86%) as a colorless liquid. ¹H-NMR (400 MHz, CHLOROFORM-D) δ 2.69-2.42 (m, 3H), 1.62-1.55 (m, 3H), 1.30-1.21 (m, 3H) ppm.

GC (DB1-1HT column, 40-360° C. constant flow method, 25° C./min, injector 250° C.) RT 2.750 min, 96.1%.

### Step 5

### Synthesis of (3R,4S,5R)-3-(3,4-difluoro-2-methoxyphenyl)-4,5-dimethyl-5-(trifluoromethyl)dihydrofuran-2(3H)-one (XXIII)

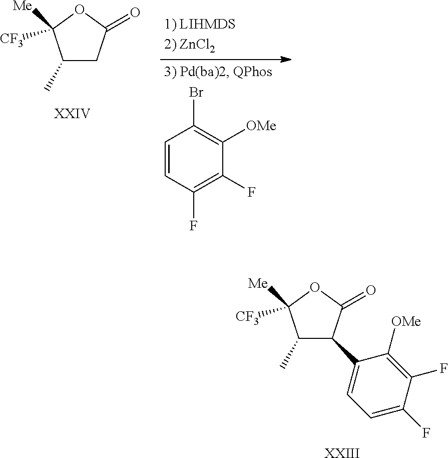

### Procedure 1

A solution of LiHMDS in tetrahydrofuran (84 mL of 1 M, 84.000 mmol) was added over 15 minutes to a solution of Compound XXIV (14.2 g, 74.064 mmol) in tetrahydrofuran (100 mL) at −50° C. under nitrogen. After 1 hour, a solution of zinc chloride in tetrahydrofuran (335 mL of 0.5 M, 167.50 mmol) was added over 40 minutes and the reaction mixture was stirred between −52 and −45° C. After 75 minutes, a solution of Pd(dba)₂ (12.880 mg, 0.0224 mmol), QPhos (1.08 g, 1.5196 mmol), and 1-bromo-3,4-difluoro-2-methoxy-benzene (11.04 g, 49.503 mmol) in tetrahydrofuran (110 mL) was added over 5 minutes and the reaction mixture was allowed to warm up to room temperature overnight. The reaction mixture was cooled down to 5° C. and 20% aqueous ammonium chloride (100 mL) was added. Methyl tert-butyl ether (100 mL) and water (50 mL) were added. The aqueous layer was diluted with water (100 mL) and extracted with methyl tert-butyl ether (100 mL). The organic layers were combined, washed with 15% aqueous sodium chloride (50 mL), dried over anhydrous sodium sulfate, filtered, and concentrated under vacuum. Toluene (100 mL) was added and the organic mixture was washed with 30% aqueous sodium chloride, dried over anhydrous sodium sulfate, filtered, and concentrated under vacuum. The residue was pre-absorbed on silica gel (25 g) using dichloromethane. Flash chromatography on silica gel (125 g) using 0-6% methyl tert-butyl ether in heptanes provided Compound XXIII (10 g, 62%) as a red solid. ESI-MS m/z calc. 324.0785, found 325.1 (M+1)+; Retention time: 2.101 minutes. The mixed fractions were combined and the solvent was removed under vacuum. The residue was pre-absorbed on silica gel (4.0 g) using dichloromethane. A second purification by flash chromatography on silica gel (40 g, 25 m) using 0-20% methyl tert-butyl ether in heptanes provided Compound XXIII (2.755 g, 17%) as a red solid. ESI-MS m/z calc. 324.0785, found 325.1 (M+1)+; Retention time: 2.101 minutes. Combined yield=79%.

¹H NMR (400 MHz, CDCl₃) δ 6.91-6.80 (m, 2H), 3.98 (d, J=2.7 Hz, 3H), 3.53 (d, J=12.0 Hz, 1H), 2.77-2.62 (m, 1H), 1.67 (s, 3H), 1.19-1.14 (m, 3H) ppm. ¹⁹F NMR (377 MHz, CDCl₃) δ −76.10 (s, 3F), −135.14-−135.71 (m, 1F), −153.19 (dd, J=19.8, 3.4 Hz, 1F) ppm.

### Procedure 2

To lithium bis(trimethylsilyl) amide in THF (1 L of 1 M, 1000.0 mmol), under argon, at −25° C., was added, with cooling, a solution of Compound XXIV (156.1 g, 857.03 mmol) in tetrahydrofuran (500 mL) so that the temperature was maintained below −25° C. On stirring at −25° C. for 0.5 h, zinc chloride in THF (3.955 L of 0.5 M, 1.9775 mol) was added so that the temperature was maintained below −25° C. On stirring at −25° C. for 0.5 h, a solution of XPhos (9.5 g, 19.928 mmol) and bis(dibenzylideneacetone) palladium(0) (7.6 g, 13.217 mmol) in tetrahydrofuran (500 mL) was added followed by a solution of 1-bromo-3,4-difluoro-2-methoxybenzene (147 g, 659.15 mmol) in tetrahydrofuran (250 mL). The reaction mixture was stirred at −25° C. for 1 h then allowed to warm to room temperature and stirred overnight. The reaction mixture was then cooled to 0° C. and saturated aqueous ammonium chloride (4 L) added. The mixture was extracted with TBME (2×1.5 L) and the combined organic extracts washed with brine (1 L), dried (sodium sulphate) and concentrated. Purification by flash chromatography on silica gel (0-5% TBME in heptane) then crystallisation from heptane (300 mL) on cooling to 10° C. gave Compound XXIII (96.1 g, 45%) as a white solid.

¹H-NMR (400 MHz, CHLOROFORM-D) δ 6.83 (m, 2H), 3.97 (m, 3H), 3.52 (d, 1H), 2.68 (m, 1H), 1.65 (m, 3H), 1.17 (m, 3H) ppm.

### Step 6

### Synthesis of (3S,4S,5R)-3-(3,4-difluoro-2-methoxyphenyl)-4,5-dimethyl-5-(trifluoromethyl)dihydrofuran-2(3H)-one (X)

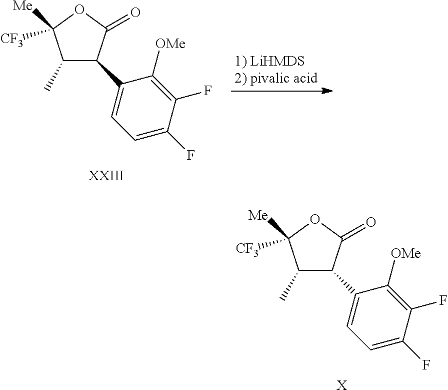

### Procedure 1:

To lithium bis(trimethylsilyl)amide in THF (480 mL of 1 M, 480.00 mmol) under argon, at −25° C., was added drop-wise a solution of Compound XXIII (100 g, 308.41 mmol) in tetrahydrofuran (500 mL). On stirring at −25° C. for 0.5 h, the solution was allowed to warm to 0° C. and stirred for 1 h. The solution was then cooled to −25° C. and added to a solution of pivalic acid (315 g, 3.0842 mol) in tetrahydrofuran (1 L), under argon, at −25° C. On stirring at −25° C. for 0.5 h, TBME (1 L) was added portion-wise so that the temperature did not exceed −15° C. followed by 2M aqueous hydrochloric acid (1.5 L), so that the temperature did not exceed 0° C. Sodium chloride (480 g, 8.2132 mol) was added and the mixture allowed to warm to room temperature. The organic layer was separated, concentrated, treated with heptane (1 L) and concentrated further. The resultant oil was dissolved in TBME (1 L), washed with saturated aqueous sodium hydrogencarbonate solution (6×1 L), then 0.5M aqueous hydrochloric acid (500 mL) then brine (500 mL), dried (sodium sulphate) and concentrated to give crude Compound X (129.3 g, 92%) as a yellow oil. The oil was contaminated with 29% pivalic acid, based on ¹H NMR.

¹H-NMR (400 MHz, CHLOROFORM-d) δ 6.89 (m, 2H), 4.49 (m, 1H), 3.99 (d, 3H), 2.88 (m, 1H), 1.70 (m, 3H), 0.79 (m, 3H) ppm

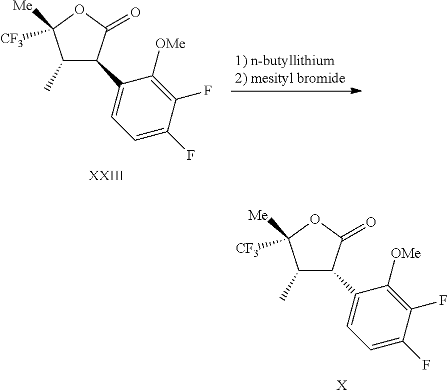

### Procedure 2:

A solution of n-butyllithium in hexanes (0.95 mL of 2.5 M, 2.3750 mmol) was slowly added to a solution of mesityl bromide (503 mg, 2.5265 mmol) in tetrahydrofuran (6.5 mL) at −78° C. and the reaction mixture was warmed up to −50° C. After stirring for 45 minutes at this temperature, the reaction mixture was cooled down to −78° C. and a solution of Compound XXIII (503 mg, 1.5498 mmol) in tetrahydrofuran (5 mL) was slowly added over 15 minutes. After 1.5 hours, a room temperature solution of salicylic acid (535 mg, 3.8734 mmol) in tetrahydrofuran (2.2 mL) was slowly added. After 30 minutes, formic acid (244.00 mg, 0.2 mL, 5.3014 mmol) was added, the reaction mixture was warmed up to room temperature and concentrated under vacuum. The residue was dissolved in methyl tert-butyl ether (20 mL) and the organic mixture was washed with 10% aqueous sodium carbonate (2×15 mL). The basic aqueous washes were combined and re-extracted with methyl tert-butyl ether (20 mL). The organic layers were combined, washed with 15% aqueous sodium chloride (15 mL), dried over anhydrous sodium sulfate, filtered and concentrated under vacuum to provide Compound X (545 mg, 74%) as a light yellow oil. ESI-MS m/z calc. 324.0785, found 325.1 (M+1)+; Retention time: 3.217 minutes.

### Example 4

Compound XXIV may alternatively be synthesized in accordance with Scheme 5.

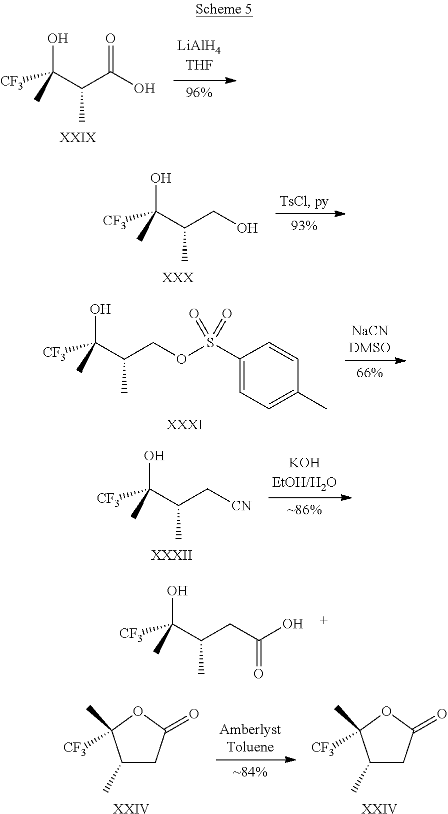

### Step 1

### Synthesis of (2S,3R)-4,4,4-trifluoro-2,3-dimethylbutane-1,3-diol (XXX)

To lithium aluminium hydride in tetrahydrofuran (2.4 L of 2.4 M, 5.7600 mol) under argon was added drop-wise, over 2 hours, a solution of (2R,3R)-4,4,4-trifluoro-3-hydroxy-2,3-dimethyl-butanoic acid (XXIX) (357.3 g, 1.9196 mol) in tetrahydrofuran (750 mL) at 45-55° C. On complete addition the mixture was heated under reflux for 0.5 h then cooled to 0° C.3. 1:1 tetrahydrofuran-water (750 mL) was added drop-wise over 1 hour at 0-20° C. 5M aqueous HCl (4.5 L) was then added drop-wise, over 40 minutes, at 0-20° C. and the product extracted into TBME (2×2 L). The combined organic extracts were washed with brine (1 L), dried (Na₂SO₄) and concentrated. The reaction was repeated 5 times and all batches combined to give Compound XXX (1934 g, 96%) as a pale yellow oil, which was used in the following step without purification. The oil was contaminated with 1.6% THF, based on ¹H NMR.

¹H-NMR (400 MHz, CHLOROFORM-D) δ 3.90-3.66 (m, 4H), 2.07 (m, 1H), 1.38 (s, 3H), 1.03 (m, 3H) ppm.

### Step 2

### Synthesis of (2S,3R)-4,4,4-trifluoro-3-hydroxy-2,3-dimethylbutyl 4-methylbenzenesulfonate (XXXI)

To a solution of Compound XXX (322.3 g, 1.8423 mol) in pyridine (1.6 L), under argon, at 0° C., was added portion-wise, over 1.5 h, p-toluenesulfonyl chloride (439 g, 2.3027 mol) at 0-5° C. On complete addition the mixture was stirred at 0-5° C. for 4 h then stirred at 10° C. overnight. The mixture was then cooled to 0° C. and treated with water (6.5 L). The mixture was extracted with TBME (2×1.5 L) and the combined organics washed with 2M aqueous HCl (2×2 L) then saturated aqueous copper sulphate solution (2 L), dried (Na₂SO₄) and concentrated. The reaction was repeated 5 times and all batches combined to give Compound XXXI (3399 g, 93%) as an orange oil, which was used in the following step without purification. The oil was contaminated with 1.6% TBME, based on ¹H NMR.

¹H-NMR (400 MHz, CHLOROFORM-D) δ 7.77 (m, 2H), 7.30 (m, 2H), 4.26 (m, 1H), 3.91 (m, 1H), 2.44 (s, 3H), 2.19 (m, 2H), 1.33 (s, 3H), 1.08 (m, 3H) ppm.

### Step 3

### Synthesis of (3S,4R)-5,5,5-trifluoro-4-hydroxy-3,4-dimethyl-pentanenitrile (XXXII)

To a solution of Compound XXXI (566.5 g, 1.7082 mol) in dimethyl sulfoxide (2.2 L) was added sodium cyanide (125.6 g, 2.5629 mol) and the reaction mixture stirred at 80° C. overnight. On cooling to room temperature, water (6.6 L) was added and the product extracted into dichloromethane (3×2 L). The combined organics were concentrated in vacuo and the resultant oil was dissolved in TBME (2 L), washed with brine (500 mL), dried (sodium sulphate) and concentrated. The reaction was repeated 5 times and all batches combined to give Compound XXXII (1371 g, 66%) as a brown oil, which was used in the following step without purification. The oil was contaminated with 10% DMSO, based on ¹H NMR.

¹H-NMR (400 MHz, CHLOROFORM-D) δ 2.89 (m, 1H), 2.14 (m, 2H), 1.28 (m, 3H), 1.15 (m, 3H) ppm, hydroxyl proton not observed.

### Step 4

### Synthesis of (4S,5R)-4,5-dimethyl-5-(trifluoromethyl)tetrahydrofuran-2-one/(3S,4R)-5,5,5-trifluoro-4-hydroxy-3,4-dimethyl-pentanoic acid (XXIV)

To a solution of XXXII (228.5 g, 1.1352 mol) in ethanol (IMS) (1.1 L) and water (1.1 L) was added potassium hydroxide (85%, 299.2 g, 4.5408 mol). The resultant solution was heated under reflux overnight. On cooling to room temperature, the ethanol was removed in vacuo and the resultant aqueous solution washed with TBME (2×500 mL)1. The aqueous solution was cooled to 0° C., acidified with cooling to pH with 36% aqueous hydrochloric acid (250 mL) at 0-4° C., extracted with TBME (3×1.5 L) and the combined extracts washed with brine (500 mL), dried (Na₂SO₄) and concentrated. The reaction was repeated 5 times and all batches combined to give Compound XXIV and (3S,4R)-5,5,5-trifluoro-4-hydroxy-3,4-dimethyl-pentanoic acid as an impurity (1122 g, ˜86%) as an orange oil, which was used in the following step without purification. The oil was contaminated with 0.5% TBME, based on ¹H NMR.

Compound XXIV was separated from (3S,4R)-5,5,5-trifluoro-4-hydroxy-3,4-dimethyl-pentanoic acid using the following procedure.

The mixture of Compound XXIV and (3S,4R)-5,5,5-trifluoro-4-hydroxy-3,4-dimethyl-pentanoic acid (561 g, ˜2.8 mol1), amberlyst 15 hydrogen form2 (10 g) and toluene (2 L) was heated under reflux for 2 h with a Dean-Stark apparatus. On cooling the reaction mixture was decanted from the resin and concentrated. The reaction was repeated and both batches combined. Vacuum distillation (102-108° C., 30 mbar) gave Compound XXIV liquid (903 g, ˜84%) as a pale yellow liquid.

¹H-NMR (400 MHz, CHLOROFORM-D) δ 2.58-2.31 (m, 3H), 1.47 (d, 3H), 1.14 (m, 3H) ppm.

GC (DB1-1HT column, 40-360° C. constant flow method, 25° C./min, injector 250° C.) RT 3.008 min, 99.8%

¹⁹F-NMR (400 MHz, CHLOROFORM-D) δ −76.4 ppm

Many modifications and variations of the embodiments described herein may be made without departing from the scope, as is apparent to those skilled in the art. The specific embodiments described herein are offered by way of example only.

1. A method of preparing a compound of formula I, or a salt thereof: 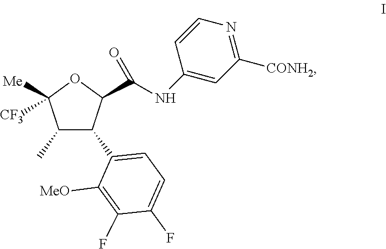 [SMILES: COc1c([C@H]2[C@H](C(=O)Nc3ccnc(C(N)=O)c3)O[C@@](C)(C(F)(F)F)[C@H]2C)ccc(F)c1F]
comprising converting a compound of formula III, or a salt thereof: 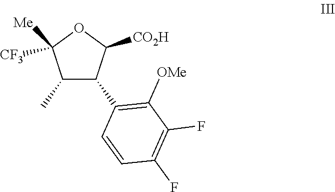 [SMILES: COc1c([C@H]2[C@H](C(=O)O)O[C@@](C)(C(F)(F)F)[C@H]2C)ccc(F)c1F]
to the compound of formula I,
optionally wherein said converting the compound of formula III to the compound of formula I comprises preparing a compound of formula IV:

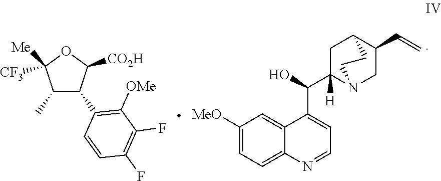 [SMILES: COc1c([C@H]2[C@H](C(=O)O)O[C@@](C)(C(F)(F)F)[C@H]2C)ccc(F)c1F ; C=C[C@H]1C[N@]2CC[C@H]1C[C@H]2[C@H](O)c1ccnc2ccc(OC)cc12]

2. (canceled)

3. The method of claim 1, wherein said converting the compound of formula III to the compound of formula I comprises reacting the compound of formula III or the compound of formula IV with a chlorinating agent to afford a compound of formula V: 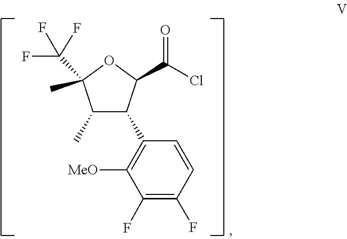
wherein the parenthetical around the compound in the compound of formula V indicates that the compound of formula V is not isolated,
optionally wherein the chlorinating agent is selected from the group consisting of phosgene, thionyl chloride, methanesulfonyl chloride, phosphorus oxychloride, phosphorus pentachloride, oxalyl chloride, isobutyl chloroformate (IBCF), pivaloyl chloride (PivCl), and diphenylphosphinic chloride (DPPCl).

4-5. (canceled)

6. The method of claim 3, wherein said converting the compound of formula III to the compound of formula I further comprising reacting the compound of formula V with a compound of formula VI: 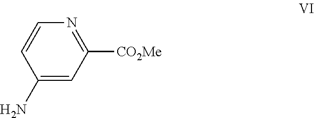 [SMILES: COC(=O)c1cc(N)ccn1]
to afford a compound of formula II:

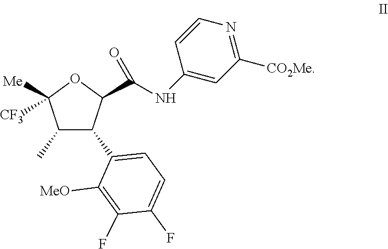 [SMILES: COC(=O)c1cc(NC(=O)[C@@H]2O[C@@](C)(C(F)(F)F)[C@@H](C)[C@H]2c2ccc(F)c(F)c2OC)ccn1]

7. The method of claim 6, wherein said converting the compound of formula III to the compound of formula I further comprises reacting the compound of formula II with ammonia to afford the compound of formula I,
optionally wherein said ammonia is in the form of a solution of ammonia in a solvent, ammonia in gas form in which an ammonia gas is bubbled into the reaction mixture, or in the form of ammonium hydroxide or an ammonium salt where ammonia is generated in situ, wherein said in situ generation of ammonia optionally comprises reacting ammonium hydroxide or the ammonium salt with an acid:
optionally wherein said reacting compound of formula II with ammonia is conducted in a solvent, wherein said solvent optionally comprises methanol, ethanol, IPA, MeCN, THF, water, or a mixture thereof.

8-11. (canceled)

12. The method of claim 1, further comprising recrystallizing the compound of formula I from a solvent system comprising acetone to afford the compound of formula I as a solid,
optionally wherein the solvent system comprises acetone and water.

13-14. (canceled)

15. The method of claim 1, further comprising hydrolyzing a cyano-compound of formula VII: 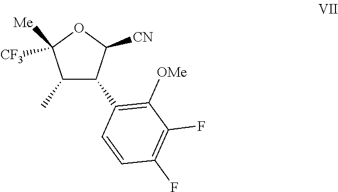 [SMILES: COc1c([C@H]2[C@H](C#N)O[C@@](C)(C(F)(F)F)[C@H]2C)ccc(F)c1F]
to afford the compound of formula III,
optionally wherein:
the cyano-compound is enzymatically hydrolyzed using a nitrilase; and/or
the hydrolysis is conducted in a solvent comprising ethanol, methanol, 1-propanol, 2-propanol, dioxane, water, THF, or a mixture thereof; and/or
the hydrolysis of the cyano-compound is conducted at about 25 to about 75° C., about 30 to about 70° C., about 35 to about 65° C., about 40 to about 60° C., about 45 to about 60° C., about 50 about 60° C., or about 55° C.

16-18. (canceled)

19. The method of claim 15, further comprising reacting a compound of formula VIII, 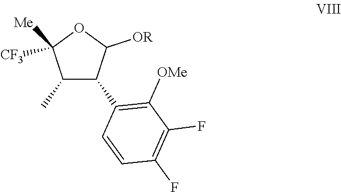
wherein OR is OC(═O)—Z, OC(═O)OZ, OC(═O)CH═CH—Z, or OP(═O)Z₂ wherein Z may be an unsubstituted aryl or an aryl substituted by CN, halo, NO₂, or a short chain alkyl, alkoxy, haloalkyl, or haloalkoxy group wherein the short chain comprises 1, 2, 3, or 4 carbon atoms;
with a cyanating agent to afford the compound of formula VII optionally wherein:
Z is C₁-C₄ alkyl or C₁-C₄ haloalkyl; or Z is phenyl and naphthyl; and/or
said cyanating agent is selected from trimethylsilyl cyanide, diethylaluminum cyanide, KCN, NaCN, TBACN, and HCN; and/or
said reaction between compound VIII and the cyanating agent is conducted in the presence of a Lewis acid, optionally wherein the Lewis acid is selected from boron trifluoride ethyl etherate (BF₃OEt₂), TiCl₄, InCl₃, AgSbF₆, iodine, ZnBr₂, Al(OiPr)₃, MgCl₂, Mn(acac)₂, MnCl₂, TMSOTf, and SnCl₄; and/or
said reaction between compound VIII and the cyanating agent is conducted in a solvent comprising toluene, dichloromethane, 2-methylTHF, acetonitrile, methanol, 1,2-dichloroethane, nitromethane, or a mixture thereof.

20-27. (canceled)

28. The method of claim 19, further comprising reacting a compound of formula IX: 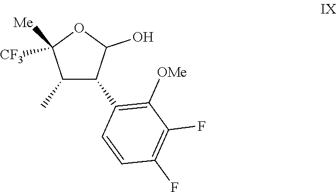 [SMILES: COc1c([C@H]2C(O)O[C@@](C)(C(F)(F)F)[C@H]2C)ccc(F)c1F]
with an acid anhydride or an acid chloride to afford the compound of formula VIII.

29. The method of claim 28, further comprising reducing a compound of formula X: 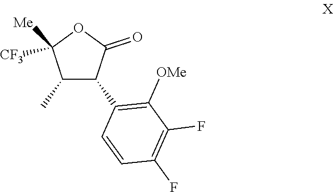 [SMILES: COc1c([C@H]2C(=O)O[C@@](C)(C(F)(F)F)[C@H]2C)ccc(F)c1F]
with a reducing agent to afford the compound of formula IX,
optionally wherein:
said reducing agent is selected from diisobutylaluminum hydride, Red-Al, NaBH₄/BF₃, titanocene with polymethylhydrosiloxane, and phenylsilane; and/or
said reducing the compound of formula X is conducted in an organic solvent or solvent mixture, optionally wherein said solvent or mixture comprises toluene, dichloromethane, 2-methyl THF, THF, TFT, MTBE, CPME, heptane, or a mixture thereof; and/or
said reducing the compound of formula X is conducted at about −78° C. to about 0° C., about −60° C. to about 0° C., about −50° C. to about −10° C., about 40° C. to about −10° C., about 30° C. to about −10° C., about −30° C. to about −15° C., about 25° C. to about −15° C., or about −20° C.; and/or
said reducing the compound of formula X is conducted in the presence of CuCl, CuI, CuTol, CuBr, CuF, Cu(II)Cl, DMAP, 2,6-lutidine, LiI, or pyridine.

30-35. (canceled)

36. The method of claim 29, further comprising an asymmetric hydrogenation of a compound of formula XI: 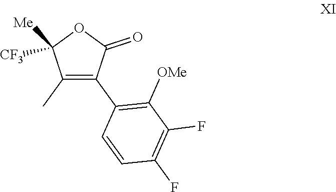 [SMILES: COc1c(C2=C(C)[C@](C)(C(F)(F)F)OC2=O)ccc(F)c1F]
to afford the compound of formula X
optionally wherein:
said asymmetric hydrogenation is conducted in the presence of a hydrogenation catalyst, wherein said catalyst is optionally selected from Pd/C, Pd/Al₂O₃, Pt/C, Ni (Raney), Co (Raney), Rh/C, Ir/C, Ru/C, Pd(OH)₂, homogeneous chiral Ru, and homogeneous chiral Rh; and/or
said asymmetric hydrogenation is conducted using a suitable hydrogen source, wherein the hydrogen source is optionally selected from H₂ gas, NiCl₂/NaBH₄ in methanol, and Et₃SiH; and/or
said asymmetric hydrogenation is conducted in the presence of H₂ gas using Pd/C as a catalyst; and/or
said asymmetric hydrogenation is conducted in an organic solvent or solvent mixture, wherein the solvent or mixture optionally comprises IPA, EtOAc, MeOH, nBuOH, THF, MTBE, CPME, IPAc, nBuAc, Toluene, Ethanol or a mixture thereof; and/or
said asymmetric hydrogenation is conducted in the presence of TFA, AcOH, H₂SO₄, H₃PO₄, MSA, Cs₂CO₃, CuCl, MgF₂, LiBr, CsF, ZnI, LiOTf, imidazole, KF, Bu₄NOAc, or NH₄BF₄.

37-45. (canceled)

46. The method of claim 36, further comprising coupling a compound of formula XIII: 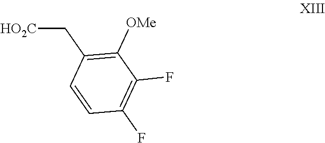 [SMILES: COc1c(CC(=O)O)ccc(F)c1F]
with a compound of formula XII:
to afford the compound of formula XI, 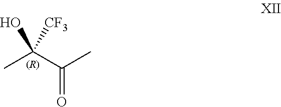
optionally wherein the coupling reaction between compounds of formulae XII and XIII is conducted in the presence of a coupling agent or a chlorinating agent, wherein:
the coupling agent is optionally selected from CDI and T3P; or
the chlorinating agent converts compound XIII to an acid chloride, which is not isolated before reacting it with the compound of formula XII, wherein the chlorinating agent is optionally selected from oxalyl chloride and thionyl chloride.

47-50. (canceled)

51. The method of claim 1, further comprising oxidation of a compound of formula XIV: 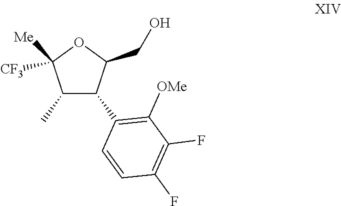 [SMILES: COc1c([C@H]2[C@H](CO)O[C@@](C)(C(F)(F)F)[C@H]2C)ccc(F)c1F]
to afford the compound of formula III,
optionally wherein:
said oxidation comprises reacting compound XIV with TEMPO in the presence of NaOCl; and/or
said oxidation is conducted in an organic solvent; and/or
said oxidation is conducted in the presence of a mild base at between about −10° C. and about 40° C., between about −10° C. and about 35° C., between about −5° C. and about 35° C., between about 0° C. and about 30° C., between about 0° C. and 25° C., about 5° C., about 10° C., about 15° C., or about 20° C.

52-54. (canceled)

55. The method of claim 51, wherein the compound of formula XIV is obtained by ring closure of a compound of formula XVI: 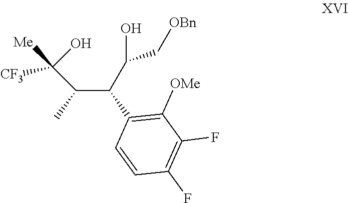 [SMILES: COc1c([C@@H]([C@H](O)COCc2ccccc2)[C@H](C)[C@@](C)(O)C(F)(F)F)ccc(F)c1F]
followed by deprotection of the resultant compound of formula XV: 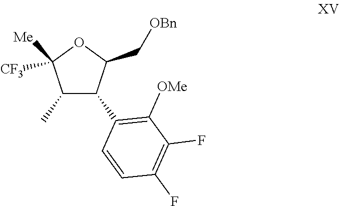 [SMILES: COc1c([C@H]2[C@H](COCc3ccccc3)O[C@@](C)(C(F)(F)F)[C@H]2C)ccc(F)c1F]
to afford the compound of formula XIV,
optionally wherein:
the ring closure reaction comprises reacting compound XVI with methanesulfonyl chloride in the presence of a non-nucleophilic base, wherein the non-nucleophilic base is optionally a tertiary amine; and/or
the ring closure reaction is conducted between about −5° C. and about 5° C.

56-58. (canceled)

59. The method of claim 55, further comprising reacting the compound of formula XV with H₂ in the presence of a Pd/C catalyst to afford the compound of formula XIV.

60-148. (canceled)

149. A compound of formula:

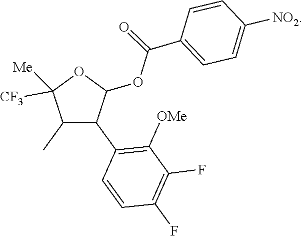 [SMILES: COc1c(C2C(OC(=O)c3ccc([N+](=O)[O-])cc3)OC(C)(C(F)(F)F)C2C)ccc(F)c1F]

150. The compound of claim 149 having the formula:

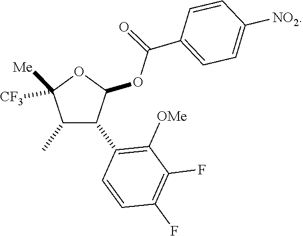

151. The compound of claim 149 having the formula:

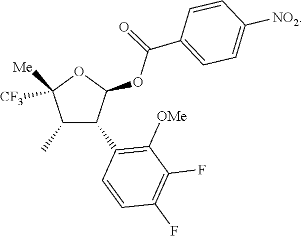 [SMILES: COc1c([C@H]2[C@H](OC(=O)c3ccc([N+](=O)[O-])cc3)O[C@@](C)(C(F)(F)F)[C@H]2C)ccc(F)c1F]

152. (canceled)

153. A compound of formula:

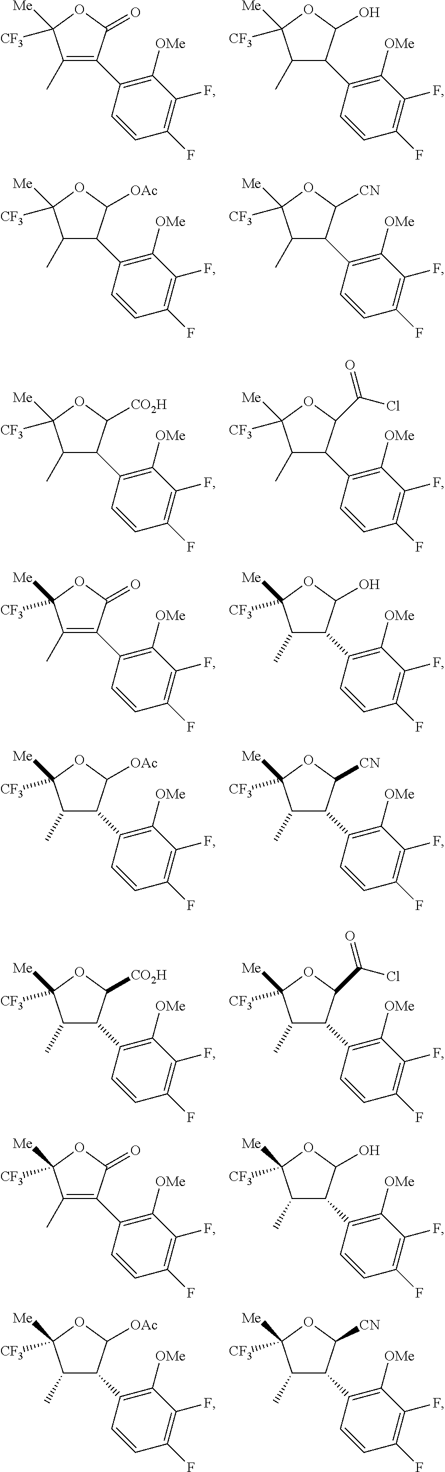

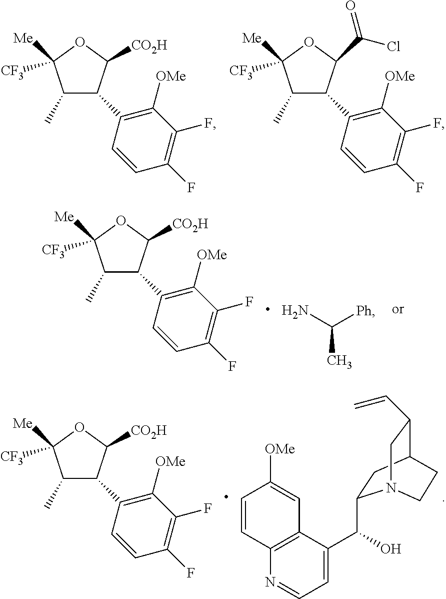

154. The compound of claim 153, wherein the compound is:

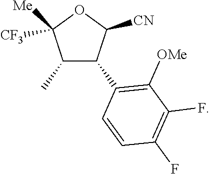

155. The compound of claim 153, wherein the compound is: [chemical structure]
